(19) United States
(12) Patent Application Publication
(10) Pub. No.: US 2009/0297523 A1
(43) Pub. Date: Dec. 3, 2009

(54) Erm family binding agents and their use in diagnosis and treatment of proliferative conditions

(75) Inventors: Frederick Naftolin, Woodbridge, CT (US); Ahmed Fadiel-Metwaly, New Haven, CT (US)

Correspondence Address: WOLF GREENFIELD & SACKS, P.C., 600 ATLANTIC AVENUE, BOSTON, MA, 02210-2206, US

(73) Assignee: Yale University, New Haven, CT (US)

(21) Appl. No.: 11/658,214

(22) PCT Filed: Jul. 27, 2005
(86) PCT No.: PCT/US05/26712

§ 371 Date: Jul. 2, 2009

Related U.S. Application Data
(60) Provisional application No. 60/591,714, filed on Jul. 27, 2004.

(51) Int. Cl. A61K 39/395; C12Q 1/02

(52) U.S. Cl. 424/138.1; 435/7.1

(57) ABSTRACT

The methods and compositions of the invention provide new diagnostic markers for cancers (e.g., ovarian cancer, PPC, etc.) and other proliferative diseases or conditions such as psoriasis and endometriosis. The methods and compositions of the invention further provide new treatments for such proliferative diseases or conditions.

### REFERENCE TO RELATED APPLICATION

This application claims the benefit of the filing date of U.S. Provisional Application Ser. No. 60/591,714, entitled “ANTIBODIES TO ERM PROTEIN BLOCK INVASIVENESS OF CANCER CELLS AND IDENTIFY THE PRESENCE AND LEVEL OF ERM PROTEINS IN PROLIFERATION CONDITIONS,” and filed on Jul. 27, 2004. The teachings of the referenced application are incorporated herein by reference.

### BACKGROUND OF THE INVENTION

Cancer is the second leading cause of death in the United States. Half of all men and one-third of all women in the US will develop cancer during their lifetime. Today, millions of people are living with cancer or have had cancer. The sooner a cancer is found and treatment begins, the better are the chances for survival.

To illustrate, ovarian cancer is cancer that begins in the cells that constitute the ovaries, including surface epithelial cells, germ cells, and the sex cord-stromal cells. The most common type of cancer arising from the ovary is ovarian epithelial cancer (OVCA). A second, less common cancer (˜10% of expected OVCA's) apparently arises from the peritoneum and has a clinical course indistinguishable from OVCA. These so-called primary peritoneal cancers (PPC) may arise from the same root tissue that covers the ovary, the peritoneum, explaining their similarity. Cancer cells that metastasize from other organ sites to the ovary (secondary lesions, most commonly breast or colon cancers) are also encountered, but are not generally considered ovarian cancers.

Even in this era of increased interest in the health needs of women, epithelial OVCA stands out as a deadly disease in special need of attention. According to the American Cancer Society, ovarian cancer accounts for only 4 percent of all cancers among women, but ranks fifth as a cause of their deaths from cancer. Over 16,000 women die each year from OVCA/PPC, making it the leading cause of death from gynecologic malignancy (Murphy et al., Sci Am. 275(3): 126-32, 1996).

This high rate of late detection of OVCA is caused by the absence of good diagnostic tests. Malignant cells can progress for a long time before they become symptomatic locally or their metastases cause symptoms. Pelvic examination is not successful in finding primary OVCA. Present markers, such as CA125, are non-specific and show false positives. The result of these failures is that OVCA is usually detected late, typically at an advanced stage when metastatic disease is the rule and the outcome is almost uniformly fatal.

In addition, few patients survive metastatic disease because no curative drug treatment exists for metastatic OVCA. Accordingly, the mortality rate attributed to OVCA has not changed significantly in the last 50 years despite the availability of new treatments (Choudhury et al., Int J Cancer. 108(1): 71-7, 2004; Hu and Xing, Curr Opin Mol Ther. 5(6): 625-30, 2003; Palm et al., J Nucl Med. 44(7): 1148-55, 2003; Iwamoto et al., Int J Gynecol Cancer. 13(1): 28-31; Argiris et al., Clin Cancer Res. 10(4): 1409-20, 2004).

Endometrial cancer (ENDOCA) is the most common form of gynecological cancer in women. It arises from the endometrial lining and, unlike OVCA, invades lymphatics and blood vessels to metastasize widely. Although ENDOCA cells are of a similar level of aggressivity as OVCA, ENDOCA is not as lethal because it is early on associated with uterine bleeding that sets off a diagnostic workup that usually exposes the ENDOCA and results in treatment before metastases occur. However, there is no simple or painless method of performing this evaluation. Usually, an endometrial biopsy is required, and thus repeated testing is not acceptable. Moreover, endometrial biopsies have a low but definite rate of false negatives.

Thus, both OVCA and ENDOCA are examples of cancers which require symptoms to reach a significant threshold for detection by present methods of diagnosis. However, by then it is often too late for OVCA treatment, and usually results in hysterectomy and infertility in the case of ENDOCA.

Thus there is a need to develop new cancer diagnosis and treatment methods, particularly those for diagnosing and/or inhibiting cancer invasion and/or metasasis, and particularly for cancers such as OVCA and ENDOCA.

### SUMMARY OF THE INVENTION

The instant invention is partly based on the discovery that expression of ERM family proteins (such as ezrin expression) is correlated with a number of indications relating to proliferative diseases, such as cancer and certain benign proliferative diseases. For example, expression of the ERM family proteins (such as ezrin) is correlated with cell motility, invasion, and metastasis. Ezrin is over-expressed in metastatic cancer to a greater extent than primary cancers, which in turn express higher levels than normal tissues. Ezrin expression levels also correlate with invasive behavior and prognosis of certain cancers, such as endometrial and ovarian cancers and cancer cells.

It is important to note that ezrin and the other ERM proteins are present at relatively low levels or even absent in most normal tissues. The expression of the ERM proteins that have high biochemical homology is tissue-dependent. ERM proteins may be co-expressed in a cell/tissue, or they may be solitary. The lower expression in normal cells than in highly proliferating cells and cancers indicates that ezrin or ERM-targeted therapies should be well tolerated, since normal cells appear to be less dependent on ERM proteins. The significant correlation of specific ERM over-expression to specific cell types, such as cancers, makes the therapeutic index of such treatment likely to be very favorable. The correlation between ERM expression and certain proliferative disease conditions also indicates that ERM expression is a useful disease marker.

The instant invention is also partly based on the discovery that antagonists to ERM family proteins, including extracellular antagonists (e.g., anti-ERM antibodies that are administered in vivo) inhibit the function of ERM in cells from ERM-associated proliferative diseases, such as cancer. For example, two different anti-ezrin monoclonal antibodies have been found to be effective in inhibiting invasion of Matrigel membranes (an accepted index of metastatic phenotype and action) and cancer cell proliferation by ezrin over-expressing cells, such as OVCA and ENDOCA. Importantly, it is also shown herein that the anti-invasive and anti-proliferative effect of anti-ezrin antiserum is dose-dependent.

The finding that anti-ezrin monoclonal antibodies bind to ezrin in live cells, and inhibit the invasive and proliferative behavior of ezrin over-expressing cancer cells under the same conditions that lead to inhibition of Matrigel membrane penetration and cell proliferation, indicates clinical usefulness of inhibiting ERM protein functions using ERM antagonists (e.g., extracellular ERM antagonists such as antibodies).

The instant invention is also partly based on the discovery that certain free-floating, ezrin-positive materials are present in culture media and in vivo-collected cell-free biological fluids that have access to the plasma membranes of ERM expressing cells. These findings are supported by microscopic examinations of such cells and tissues showing the budding off of processes from ezrin-expressing cells, e.g., OVCA, ENDOCA and normal endometrial cells. These findings indicate that ERM proteins and/or such free-floating ERM-containing structures, which may be present in certain body fluids such as ascetic fluid, endometrial secretion, blood, urine and endometrial washings from individual women or men (ERMS are expressed in male cancers such as prostate cancer), can serve as clinical tumor markers. In other words, the discovery of the relationship of ERM proteins to membranes, and the breaking off or shedding of ERM-containing cell surface structures, make certain ERM protein binding agents useful in detecting and/or measuring the presence and/or progress of certain proliferative diseases, and/or monitoring the effects of treatments based on detection of ERMs in bodily fluids.

“Binding agents” for the ERMs include those that bind or interact with at least one ERM family proteins, but does not include antisense polynucleotides against a polynucleotide encoding the ERM family proteins.

Thus in one aspect, the invention provides a method of inhibiting a proliferative condition in an individual, comprising administering to the individual an effective amount of a binding agent which binds an ERM family protein.

In a related aspect, the invention provides for the use of an effective amount of an ERM binding agent for the formulation of a medicament, particularly for the formulation of a medicament for the treatment of a proliferative disorder such as cancer or other proliferative disorders as described herein.

“Inhibit” as used herein includes completely stalling a biological activity (such as cell proliferation, invasion, or metastasis, etc.), preventing or at least delaying the onset of a biological process, and/or reducing the severity and/or symptoms of a biological condition, etc.

The methods of the invention can be used to treat or prevent many proliferative diseases or conditions associated with ERM family proteins. As used herein, “proliferative diseases or conditions” include conditions with excessive cell proliferation and/or cell number increase. For example, the proliferative condition includes cancer, such as ovarian cancer, endometrial cancer (ENDOCA), endometrial adenocarcinoma (such as uterine endometroid adenocarcinoma or “UEC”), primary peritoneal cancer (PPC), renal adenocarcinoma, brain hemangioblastoma, pancreatic adenocarcinoma, epidermoid carcinoma, osteosarcoma, epithelial cancer, melanoma, squamous skin carcinoma, leukemia, breast cancer, glioblastoma, schwannoma, meningioma, malignant mesothelioma, neurofibromatosis, colon cancer, oral cancer, or rhabdomyosarcoma, or other cancers selected from the group consisting of lung cancer, prostate cancer, pancreatic cancer, leukemia, liver cancer, stomach cancer, uterine cancer, testicular cancer, brain cancer, non-hodgkin's lymphoma, hodgkin's lymphoma, Ewing's sarcoma, osteosarcoma, neuroblastoma, rhabdomyosarcoma, melanoma, and brain cancer.

In addition, the cancer may be invasive and/or metastatic. In that case, the binding agent of the invention may inhibit cancer invasion and/or metastasis.

In certain embodiments, the binding agent of the invention also inhibits cell proliferation.

Alternatively, the methods of the invention may be used to treat other proliferative conditions, such as certain benign proliferative disorders. These proliferative conditions may include tuberosclerosis, psoriasis, endometriosis, complex endometrial hyperplasia (cH), atypical endometrial hyperplasia (aH), polyps (such as colon polyps), or neurofibromatosis.

The methods of the invention may be used to treat any individual, including a human patient or a non-human mammal, such as laboratory animals (mouse, rat, hamster, rabbit, and other rodents), farm animals (sheep, goat, horse, pig, cattle, etc.), or pets (cat, dog, etc.).

Various ERM protein binding agents may be used in the instant invention. For example, the binding agent may be an antibody, or a functional fragment thereof. “Functional fragment” includes a fragment that binds the antigen, preferably binds the antigen and has at least one functional effect of the full antibody (such as inhibit the function of the antigen or binding partner), especially when used in the context of the subject treatment method. However, functional fragment may only need to be able to bind its intended target molecule for the various diagnosis embodiments of the invention. The antibody may be a polyclonal antibody or a monoclonal antibody. The antibody may be a xenogeneic, an allogeneic, or a syngeneic antibody. The antibody can also be a modified antibody selected from the group consisting of: a chimeric antibody, a humanized antibody, and a fully human antibody. The functional fragment of an antibody may be F(ab′)2, Fab, Fv, or scFv, one or more CDR's, etc.

In addition, the binding agent can be a small molecule antagonist of the ERM proteins, such as those with molecular weights no more than about 5000 Da, 4000 Da, 3000 Da, 2000 Da, 1000 Da, 500 Da, 200 Da, or less than 100 Da. Such small molecule binding agents may be small peptides, or peptidio-mimetics, or any other organic or inorganic compounds that can bind any ERM protein and inhibit ERM protein function (such as their role in proliferation and/or invasion, metastasis).

The binding agents of the invention may be specific for only one of the ERM family proteins, or may be specific for a subset of all ERM family proteins, or may be pan-generic to most or all of the ERM family proteins.

The binding agents of the invention may recognize the full-length ERM protein, or recognize only a portion/fragment of the ERM protein. For example, the binding agent may bind to the N-terminal portion of the ERM protein, e.g., the portion used for ERM-binding to cell-surface receptors, such as EGFR. Binding to the ERM N-terminal domain may further inhibit the interaction between the ERM protein with its normal binding partners, such as a cell surface receptor, e.g., an EGF family receptor (e.g., EGFR or c-erbB2), an IGF family receptor, an Estrogen Receptor, an IL-1a receptor, CD43, or CD44.

One or more of the binding agents of the invention recognizing a non-overlapping or overlapping region of the same or different ERM family proteins may be used simultaneously and/or sequentially.

The binding agents of the invention may recognize both the “closed” (“dorman” or inactive version of the ERM protein that is released from the protein-synthetic machinery of the cell) and the “open” (or activated) version of the ERM protein, or only one form but not the other.

One representative member of the ERM family proteins is ezrin. Other members of the ERM family proteins include moesin, radixin, or NF2 (Neurofibromatosis Factor 2)/merlin/schwannomin (SCH). Other ERM-related proteins may include protein 4.1 and talin. These proteins may be represented in the cell or free-floating ERM-containing structures as “wild type” proteins, or modified proteins, such as those found in OVCA and other cancers, and deposited in the GenBank (see below). These may also include NF2, which in cases of neurofibromatosis is a mutated ERM and lacks a portion of the molecule that ceases its proliferation-regulating function.

The method of the invention may additionally comprise administering a second therapeutic agent that is effective against the proliferative disease or condition, sequentially or concurrently with the ERM binding agent

For example, where the proliferative condition is cancer, the second therapeutic agent may be selected from the group consisting of: methotrexate, amsacrine, azacytidine, bleomycin, busulfan, capecitabine, carboplatin, carmustine, chlorambucil, cisplatin, cladribine, cyclophosphamide, cytarabine, dactinomycin, daunombicin, decarbazine, docetaxel, doxorubicin, epirubicin, estramustine, etoposide, floxuridine, fludarabine, fluorouracil, gemcitabine, hexamethylmelamine, idarubicin, ifosfamide, irinotecan, lomustine, mechlorethamine, melphalan, mercaptopurine, mitomycin C, mitotane, mitoxantrone, oxaliplatin, paclitaxel, pemetrexed, pentostatin, plicamycin, procarbazine, ralitrexed, semustine, streptozocin, temozolamide, teniposide, thioguanine, thiotepa, topotecan, trimitrexate, valrubicin, vincristine, vinblastine, vindestine, vinorelbine, aminoglutethimide, anastrozole, asparaginase, bcg, bicalutamide, buserelin, campothecin, clodronate, colchicine, cyproterone, dacarbazine, dienestrol, diethylstilbestrol, estradiol, exemestane, filgrastim, fludrocortisone, fluoxymesterone, flutamide, genistein, goserelin, hydroxyurea, imatinib, interferon, ironotecan, letrozole, leucovorin, leuprolide, levamisole, medroxyprogesterone, megestrol, mesna, nilutamide, nocodazole, octreotide, pamidronate, porfimer, raltitrexed, rituximab, suramin, tamoxifen, temozolomide, testosterone, titanocene dichloride, trastuzumab, tretinoin, vindesine, HERCEPTIN® and other antibody therapeutics, and an anti-sense or RNAi agent against one or more genes promoting the progression of the cancer.

Furthermore, the individual receiving the treatment may be additionally subjected to radiation therapy and/or surgery, with or without continuing or re-starting the anti-ERM therapy.

Another aspect of the invention provides a method of diagnosis for, or aiding in the diagnosis of, a proliferative disorder in an individual, comprising determining the amount and/or concentration of an ERM family protein in a sample from an individual suspected of having, or at risk of having, the proliferative disorder, wherein an amount and/or concentration of the ERM family protein that is significantly higher than a normal or control sample is indicative of the existence of the proliferative condition in the individual. The method may similarly be used to diagnose other disorders associated with over-expression of ERM proteins.

The method may use a sample such as a body fluid from the individual. Many different types of body fluids may be used, including peritoneal fluid, ascitic fluid, endometrial secretion, blood, serum, urine, semen, or lymph fluid.

The assay may be done in various ways, including an Enzyme Linked ImmunoSorbant Assay (ELISA), in which a first immobilized binding agent (e.g., immobilized on a solid surface such as a 96-well plate, etc.) is used to bind and isolate an ERM protein in a fluid, and a second detection binding agent (such as a binding agent labeled by a fluorescent dye, an enzyme, or a radio label) is used to bind the bound ERM protein. The presence and amount of the labeled second detection binding agent may then be determined/measured.

According to the subject method, the amount and/or concentration of the ERM family protein detected in the sample is proportionally indicative of the severity and/or extent of the proliferative condition.

In certain embodiments, the amount and/or concentration of the ERM family protein may be used along with the results of one or more other diagnostic tests, such as those selected from the group consisting of: mammography, an early mammography program, a frequent mammography program, a biopsy procedure using a tissue of the individual, an ultrasound analysis of a suspected disease organ and optionally a normal organ, a magnetic resonance imaging (MRI) analysis of a suspected disease organ and optionally a normal organ, an electrical impedance (T-scan) analysis of a suspected disease organ and optionally a normal organ, ductal lavage, a nuclear medicine analysis (e.g., scintimammography), sequence analysis of one or more disease-associated genes (e.g., BRCA1 and/or BRCA2, etc.), and a thermal imaging of a suspected disease organ and optionally a normal organ.

The diagnosis method of the invention can be used for diagnosis of a variety of proliferative diseases/conditions, including cancer. The cancer may be ovarian cancer, endometrial cancer (ENDOCA), endometrial adenocarcinoma (such as uterine endometroid adenocarcinoma or “UEC”), primary peritoneal cancer (PPC), renal adenocarcinoma, brain hemangioblastoma, pancreatic adenocarcinoma, epidermoid carcinoma, osteosarcoma, epithelial cancer, leukemia, breast cancer, glioblastoma, schwannoma, meningioma, malignant mesothelioma, neurofibromatosis, colon cancer, oral cancer, or rhabdomyosarcoma, etc. In addition, the cancer may be invasive and/or metastatic, or may be benign.

The diagnosis method of the invention can also be used for diagnosis of benign proliferative disorders, such as tuberosclerosis, psoriasis, endometriosis, endometrial or other tissue hyperplasia, complex endometrial hyperplasia (cH), atypical endometrial hyperplasia (aH), polyps (such as colon polyps), or neurofibromatosis. Such conditions include paoriasis, or endometriosis.

The diagnosis method of the invention can be used for diagnosis in a human or a non-human mammal, such as laboratory animals (mouse, rat, hamster, rabbit, and other rodents), farm animals (sheep, goat, horse, pig, cattle, etc.), or pets (cat, dog, etc.).

The diagnosis method of the invention may be performed, e.g., the amount and/or concentration of the ERM family protein is determined, using a binding agent which binds the ERM family protein. The binding agent may be an antibody, or a functional fragment thereof. “Functional” may only require the ability to bind in the context of the subject diagnosis methods. The antibody may be a polyclonal antibody or a monoclonal antibody. The antibody may be a xenogeneic antibody, an allogeneic antibody, or a syngeneic antibody. The antibody may be a modified antibody selected from the group consisting of: a chimeric antibody, a humanized antibody, and a fully human antibody. The functional fragment may be F(ab′)2, Fab, Fv, scFv, or one or more CDR's.

In addition, the binding agent may be a small molecule with molecular weight no more than about 5000 Da, 4000 Da, 3000 Da, 2000 Da, 1000 Da, 500 Da, 200 Da, or less than 100 Da. Such small molecule binding agents may be small peptides, or peptidio-mimetics, or any other organic or inorganic compounds that can bind ERM protein.

In certain embodiments, the binding agent may be immobilized on, for example, a solid support. For instance, the binding agents may be arranged in a spacially resolved pattern in a binding agent array.

In certain embodiments, the binding agent may also be tagged by a label, such as a fluorescent label, an enzyme label, or a radio-label.

The diagnosis methods of the invention may be used to detect all ERM family proteins. A representative ERM family protein is ezrin. Other ERM family proteins may include moesin, radixin, or NF2 (Neurofibromatosis Factor 2)/merlin/schwannomin (SCH). Additional ERM-related proteins may include protein 4.1 and talin.

Alternatively, the binding agents of the invention may be specific for only one of the ERM family proteins, or may be specific for a subset of all ERM family proteins, or may be pan-generic to most or all of the ERM family proteins.

The binding agents of the invention may recognize the full-length ERM protein, or recognize only a portion/fragment of the ERM protein. For example, the binding agent may bind to the N-terminal portion of the ERM protein, e.g., the portion used for ERM-binding to cell-surface receptors, such as EGFR. Binding to the ERM N-terminal domain may further inhibit the interaction between the ERM protein with its normal binding partners, such as a cell surface receptor, e.g., an EGF family receptor (e.g., EGFR or c-erbB2), an IGF family receptor, an Estrogen Receptor, an IL-1α receptor, CD43, or CD44.

One or more of the binding agents of the invention recognizing a non-overlapping or overlapping region of the same or different ERM family proteins may be used simultaneously and/or sequentially.

The binding agents of the invention may recognize both the “closed” (“dormant” or inactive version of the ERM protein that is released from the protein-synthetic machinery of the cell) and the “open” (or activated) version of the ERM protein, or only one form but not the other.

Yet another aspect of the invention provides a complex comprising an ERM family protein binding agent bound to an extracellular ERM protein, wherein the extracellular ERM protein is on or near the extracellular plasma membrane surface of a cell, such as a cancer cell, or a benign proliferative cell, especially if it is a precancerous lesion. The cancer cell may be invasive and/or metastatic. In certain embodiments, the ERM protein of the complex may be within a free-floating ERM-containing cell surface structure.

In certain embodiments, the ERM family protein binding agent may be labeled by a moiety, such as a fluorescent dye, an enzyme, or a radio-imaging reagent.

Yet another aspect of the invention provides an in vivo complex comprising an ERM family protein binding agent bound to an ERM protein.

The in vivo complex may be formed by administering the ERM family protein binding agent to an individual having the extracellular ERM protein. In this embodiment, the individual may be a patient suffering from a cancer or a proliferative disorder.

Alternatively, the in vivo complex may be formed in vitro, and is then administered to an individual as a pharmaceutical composition. In this embodiment, the individual may be healthy, and the complex may confer prophylactic benefits to the individual. The individual may also be a patient suffering from a cancer or a proliferative disorder associated with ERM family proteins, or an individual having substantial risk of suffering from a cancer or a benign proliferative disorder associated with ERM family proteins.

The pharmaceutical composition of the invention may also comprise a pharmaceutically acceptable salt, excipient, and/or carrier for in vivo administration to an individual.

Yet another aspect of the invention relates to a method of treatment or prevention for a proliferative condition in an individual, comprising administering to the individual an effective amount of a complex comprising an ERM family protein binding agent bound to an extracellular ERM protein.

In one embodiment, the method reduces or eliminates metastatic spread of the cancer.

It should be understood that, to illustrate the claims, Applicants have concentrated on the role of one ERM family protein (e.g., ezrin) in two representative cancers (e.g., ovarian cancer and endometrial cancer). However, the compositions and methods of the invention extend further to include other normal and abnormal ERM family proteins, and/or other proliferative disorders, and the effects of classes of antagonists or binding agents that include antibodies, and functional portions as diagnostic and/or therapeutic agents.

The embodiments described above, including those described under different aspects of the invention, are contemplated to be applicable for all aspects of the inventions wherever appropriate.

### BRIEF DESCRIPTION OF THE DRAWINGS

FIG. 1 is a modified drawing of the cellular location and molecular sites of action of ezrin. Note that the folded, dormant protein that leaves the Golgi apparatus is moved to the cell membrane and unfolds there, placing its N-terminus in the area of the cell membrane. The activated ERM is shown binding (by phosphorylation) to the transmembrane/intracellular domain of the epithelial growth factor receptor (EGFR). The result is the failure of development of the metastatic phenotype and blocked cell division. For elucidation and as a comparison with a narrower, but successful and theoretically similar approach, the humanized anti-EGFR anti-cancer immunotherapy drug HERCEPTIN® is depicted as binding to the extra-cellular domain of the EGFR.

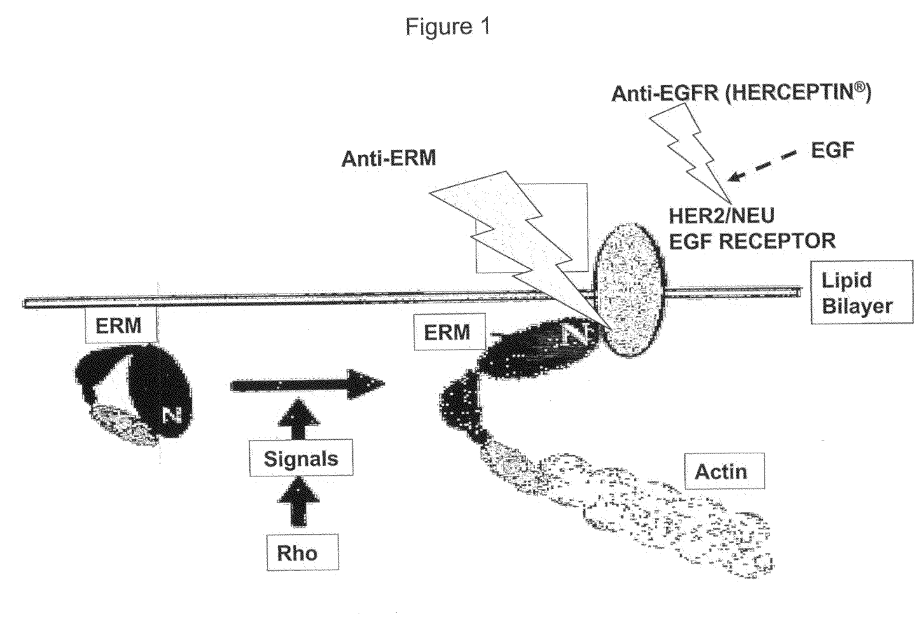

FIG. 2 shows the effects of ERM proteins on cell morphology, and illustrates the variety and number of sites that can be attacked by the ERM-binding agent, in comparison to the possible number of sites attacked by HERCEPTIN®.

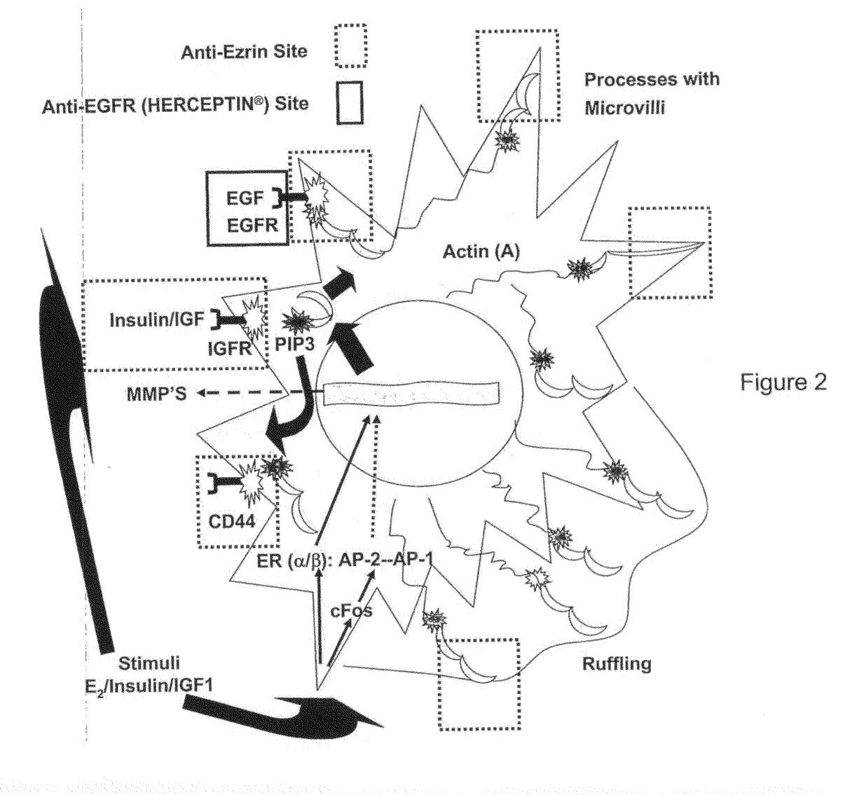

FIG. 3 shows the result of a Western blot analysis, indicating that ezrin over-expression is related to the stage of OVCA progression. The “*” and “**” represent statistically significant differences.

FIG. 4A shows the result of Matrigel invasion assays, indicating that anti-ezrin antibody administered to live endometrial cancer cells in culture inhibits invasive behavior of the cancer cells,

FIG. 4B shows that this action occurs in a dose-responsive manner. Note that in FIG. 4B, the results are presented as % inhibition of Matrigel penetration.

FIG. 5 illustrates how Matrigel membrane penetration by OVCA cells is inhibited in a dose-dependent manner by anti-ezrin monoclonal antibody. The three data points represent decreasing dilutions of monoclonal antibodies (mAb) at 1:1000, 1:500, and 1:100 dilutions. The vertical bars represent relative numbers of OVCA cells following 24-hour treatment with anti-ezrin nAb (1:1000 or 1:500 dilutions) or control untreated cultures.

FIG. 6 shows that anti-ezrin mAb also inhibits OVCA cell growth (e.g., increase in cell number) by the same starting cells that are in the above figures. These cells are grown separately, in flasks, to assess cell growth or increase in cell number. The number of cells in the flasks at the end of a 24-hour period are counted. The results from cells treated with 1:1000 or 1:500 dilutions of anti-ezrin mAb are compared with that of the control untreated cultures. At the end of the experiment, the control group had four times as many cells as the 1:500 dilution of anti-ezrin mAb (e.g., about 75% inhibition). The decrease in cell number in mAb-treated culture may be due to inhibited cell proliferation or increased apoptosis, or both.

FIG. 7 shows that anti-ezrin antibody penetrates a caveolus membrane to bind its target. It is the immunolabeling of ezrin by anti-ezrin antibody (Ab) administered to live OVCA cells in culture. The labeling is performed after the anti-ezrin Ab was added to the OVCA cell culture for five minutes, after which the cells were washed off antibodies, and then fixed and labeled by the diaminobenzidine method that labels antibodies to the anti-ezrin immunoglobulin with electron-dense material. This is the black matter seen within the cell membranes of the OVCA cells. This has occurred in a typical caveolus, which is where most of the actions ascribed to ERM proteins occur.

FIG. 8 shows Western analyses of cell-free ascetic fluid from patients with metastatic OVCA. Note the heavy band that indicates ezrin, which is about 80 kDa against the molecular sizing markers in the first lane. Note also that there are subsidiary bands that may indicate the presence of fragments of ezrin and/or variants of ezrin protein produced by the cancer cells. Several such variants have been deposited in the GenBank (see below).

### DETAILED DESCRIPTION OF THE INVENTION

Cancer is an abnormal state in which uncontrolled proliferation of one or more cell populations interferes with normal biological functioning. The proliferative changes are usually accompanied by other changes in cellular properties, including reversion to a less differentiated, more developmentally primitive state; the ability to invade surrounding tissues/organs (e.g., invasion); and/or the ability to metastasize to distant tissues/organs (metastasis).

The development of cancer consists of multiple, sequential, and interrelated steps that lead to the generation of an autonomous clone with aggressive growth potential. These steps include sustained growth and unlimited self-renewal through a process of autonomous growth signals, decreased sensitivity to growth-suppressive signals, and resistance to apoptosis. Genetic or cytogenetic events that initiate aberrant growth sustain cells in a prolonged “ready” state by preventing apoptosis.

In addition to the loss of regulated growth, another feature of many tumors is invasion of surrounding tissues. Local invasive infiltration, as in OVCA, and escape to implant in distant sites, as in ENDOCA, are key and deadly features in many tumors. These actions are accompanied by remodeling of the local vasculature and destruction of surrounding normal tissues. In addition to the ability of malignant cells to form thin, clawing processes (ruffles and protrusions) and invade physically between cells, the invasive character of certain tumors appears to depend partly on remodeling of the surrounding extracellular matrix, such as by the proteolytic destruction of the extracellular matrix components. One class of enzymes that is upregulated in certain tumors are the matrix metalloproteinases (MMPs), zinc dependent proteolytic enzymes, that cleave extracellular matrix (collagen, laminin, fibronectin, etc) as well as non-matrix substrates (growth factors, cell surface receptors, etc). The deregulation of MMPs is involved in many diseases, such as tumor metastasis, rheumatoid arthritis, and periodontal disease.

The ERM family of proteins are well-known as intracellular membrane-actin cross-linking proteins. Ezrin (also known as Cytovillin or Villin-2) is a prototypical member of the ERM protein family. Like its family members, ezrin is an important signal transduction protein that undergoes phosphorylation and translocation on stimulation by growth factors and other agents, such as estrogen. The chief partners in these actions are the contractile protein actin, that forms the cytoskeletal basis for the normal cell specialization and metastatic cell phenotype that are induced by ERM proteins, and proteins that are associated with the cell membrane, such as cell surface receptors, ERFR, IGF1R, etc. By linking these two types of proteins, the ERM proteins are indispensable to a variety of cellular functions, such as cell surface specialization, cell division, adhesion, migration, and the organization and function of cell surface structures, as described herein in regard to cancer cells.

In the context of cancer and other benign proliferative conditions, ERM proteins play roles in all steps of cancer progression (e.g., sustained growth, unlimited self-renewal, decreased sensitivity to growth-suppressive signals, and resistance to apoptosis, etc.). Overexpression of ERM proteins is found in many different cancers and benign proliferative conditions. One of the effects of ERM proteins in cancer and benign proliferative conditions may be to foster cell division, in part by the manipulation of the actin cytoskeleton. The over-expression of the ERM proteins needed for this action also underlies the potential usefulness of ERM proteins as tumor markers. In addition, Applicants have shown that development of ruffles and protrusions is accompanied by MMP2 expression in OVCA cells that are increasing their invasive activities.

The ERM family of proteins are linked to the intracellular membrane-actin via phosphorylation at the “C” and “N” termini of the activated ERM molecule. The C terminus links to actin or forms polymers with other ERM proteins; while the N-terminus is closest to the membrane, and it links to the transmembrane or cytoplasmic domain of other proteins via specific tyrosine phosphorylation. The latter linkage may require binding to spacer molecules known as ezrin/moesin-binding protein 50 (EBP50, also known as the sodium-hydrogen transporter molecule). Since they are among the most common transmembrane receptor molecules, growth factor ligation and signal transduction that triggers auto-phosphorylation of the receptor is among the most common sequence of events leading to binding and action of the ERM proteins. The example of EGFR ligation and its subsequent binding to ezrin is mentioned herein, because of the clinical relevance of the success of the humanized antibody to EGFR HERCEPTIN® that depends on indirect blockade of the ERM signal transduction pathway for its anti cancer action. The ERM proteins are involved in a variety of cellular functions, such as cell adhesion, migration, and the organization of cell surface that concern specialization such as brush borders, dense junctions, and membrane specializations necessary for invasive behavior by normal and malignant cells. Since proliferating cells must divide, the ERM proteins are necessary for cell division and proliferation. The failure of cell division is often followed by cell death (apoptosis). The loss of the C-terminus of NF2 results in cell proliferation without apoptosis, leading to neurofibromatosis. Applicants have shown the hierarchical expression of ERM proteins in cells that are undergoing proliferation, such that more ERM proteins are produced in more rapidly growing cells, such as cancer cells, proliferative phase endometrial gland cells and others. While not wishing to be bound by any particular theory, these cells have fragile specialized structures (such as microvilli and protrusions) that have the ERM proteins in high concentration, fragments of these structures are constantly breaking off into the intercellular space or to body cavities, from which they access the circulation or the body fluids (such as peritoneal fluids, ascitic fluids, endometrial secretions, blood, semen, or urine, etc.).

Therefore, the instant invention is partially based on the discovery that ERM family proteins are markers for proliferative disorders. This is based on the over-expression of ERM family proteins by cancers (including OVCA, ENDOCA, and PPC) and benign proliferative conditions. Fragmentation of the ERM-laden cell specializations (protrusions, ruffles, microvilli, etc.) releases ERM's into tissues or body cavities, and thereby into various biological fluids. For example, the presence of the ERM's is indicated by the presence of ezrin in cell-free ascitic fluid from OVCA patients. Fragments of cells with densely bound ERM proteins can furnish ERM proteins as marker proteins for diagnosis and evaluation of treatment effectiveness. Thus quantitative and qualitative tests and measurement of one or more ERM proteins, including altered ERM's found in normal cells and/or cancers, constitute valuable diagnosis/prognosis/monitoring assays for ERM-associated conditions.

The instant invention thus provides a method to use ERM binding agents as detection agents for detecting and/or quantitating the ERM proteins in a number of pathological conditions, using samples such as body fluids (e.g. peritoneal fluid, ascitic fluid, endometrial secretion, blood, serum, urine, semen, lymph fluid, etc.) obtained from an individual suffering from such conditions, or at risk of developing such conditions. One of the main advantages of the subject method is specifically due to the lack of ezrin expression by normal tissues. For example, normal ovary only expresses ezrin in the depths of the ovarian clefts and in areas where ovarian adhesions have formed. Therefore, sensitivity and specificity of the subject method are high, while false positive rate is low.

As a result, the subject method can detect a low level of true positive signal, thus providing a method for early detection and diagnosis of diseases where early diagnosis is critical for prognosis.

The subject diagnosis method not only provides an early diagnosis/screening means for certain proliferative diseases, but also provide a non-invasive means to monitor the progress of the disease over time, its responsiveness to various treatments, and/or the possible recurrence of diseases previously in remission. Thus the term “diagnosis” includes not only the initial diagnosis, but also the monitoring of disease progress, the response of the disease to specific treatment regimens, the detection of possible recurrence, and screening healthy individuals or individuals at high risk of developing the subject disease conditions, etc.

The instant invention is also partially based on the discovery that ERM binding agents (e.g., antibodies) can effectively be used in treatment of cancers, pre-cancers, and proliferative disease and like conditions. This is based on at least two findings. First, Applicants have shown that anti-ezrin antibodies administered to live cells bind ezrin. This is substantiated by morphological proof. Second, Applicants observed that, following the administration of (two different) anti-ezrin antibodies, there is a dose-dependent inhibition of two biological actions, e.g., Matrigel membrane penetration and cell growth (cell number increase). Thus Applicants have unequivocally show that ERM binding agents, such as antibodies, can inhibit the actions of ezrin.

These circumstances for the first time demonstrate that ERM proteins can be self-anchored within the plasma membrane, and that such ERM proteins are accessible to ERM family protein binding agents, such as anti-ERM antibodies. Most importantly, Applicants demonstrate for the first time that ERM binding agents, such as ERM antibodies administered to live cells effectively inhibit ERM-associated invasion and/or metastatic behavior. Thus the invention provides a method to treat or prevent a number of proliferative conditions, such as cancer or other proliferative conditions, comprising administering to an individual in need of such treatment an effective amount of an ERM binding agent as therapeutic agents.

Various aspects of the instant invention are described in more detail below.

### Exemplary ERM Proteins

Ezrin also known as Cytovillin or Villin 2, is known as a microvillar cytoplasmic peripheral membrane protein that is expressed strongly in placental syncytio-trophoblasts and in certain human tumors. It is also a component of the microvilli of intestinal epithelial cells that serves as a major cytoplasmic substrate for certain protein-tyrosine kinases.

The so-called “ERM proteins,” ezrin, radixin, and moesin, act as linkers between the plasma membrane and the actin cytoskeleton. They are involved in a variety of cellular functions, such as cell adhesion, migration, and the organization of cell surface structures. They are highly homologous, both in protein structure and in functional activity, with merlin/schwannomin, the NF2 tumor suppressor protein. The genomic structures of ezrin and moesin are highly conserved, suggesting that they diverged recently (Majander-Nordenswan et al., Hum. Genet. 103: 662-665, 1998).

Ezrin is a highly charged protein with an overall pI of 6.1 and a calculated molecular weight of about 69,000, and runs on a gel at about 80 kDa as compared to a molecular weight size standard. Highest ezrin expression was found in intestine, kidney, and lung. The ezrin cDNA clone hybridized to DNAs from widely divergent organisms, indicating that the sequence is highly conserved throughout evolution. Within its N-terminal domain, ezrin also showed a high degree of similarity of amino acid sequence to the erythrocyte cytoskeletal protein band 4.1.

Moesin stands for membrane-organizing extension spike protein (Lankes et al., Biochem. J. 251: 831-842, 1988). It was first isolated from bovine uterus, and further studies indicated that it shares a significant sequence homology to ezrin, protein 4.1, talin, radixin, and merlin. These proteins constitute a family with structural and probably functional relationships; all of them are localized to the submembranous cytoskeleton. Moesin is widely expressed in different tissues in cells, where it is localized to filopodia and other membranous protrusions that are important for cell-cell recognition and signaling and for cell movement.

Lankes and Furthmayr (Proc. Nat. Acad. Sci. 88: 8297-8301, 1991) cloned and sequenced the complete cDNA of moesin, which contains no apparent signal peptide or transmembrane domain.

Radixin functions as a membrane-cytoskeletal crosslinkers in actin-rich cell surface structures and is thereby thought to be essential for cortical cytoskeleton organization, cell motility, adhesion and proliferation. Cloning of the murine and porcine radixin cDNAs demonstrated a protein highly homologous to ezrin and moesin. Wilgenbus et al. (Genomics 16: 199-206, 1993) cloned and sequenced the human radixin cDNA and found the predicted amino acid sequence for the human protein to be nearly identical to those predicted for radixin in murine and porcine, indicating that this family of proteins are highly conserved across species.

Radixin is a modular polypeptide consists of a long, central helix, termed the alpha-domain, which connects an N-terminal 4.1/ezrin/radixin/moesin (FERM) domain required for membrane binding and a C-terminal region that contains a major actin-binding motif. Conformational regulation of radixin protein function occurs by association of the FERM and C-terminal domains, whereby the membrane- and actin-binding activities are mutually suppressed and the protein is thought to take an inactive “closed” form (Hoeflich and Ikura, Int J Biochem Cell Biol. 36(11): 2131-6, 2004).

Myosin regulatory light chain interacting protein (MIR) also belongs to the ezrin, radixin, moesin (ERM) family of proteins (Bonhauser et al., FEBS Lett. 553(1-2): 195-9, 2003).

The ERM family proteins includes mammalian and non-mammalian homologs. The human ezrin protein sequence is available in the NCBI database as NP_{—}003370 (nucleic acid sequence NM_{—}003379.3). The human moesin protein sequence is available in the NCBI database as NP_{—}002435 (nucleic acid sequence NM_{—}002444.2). The human radixin protein sequence is available in the NCBI database as NP_{—}002897 (nucleic acid sequence NM_{—}002906.3). The human NF2 protein sequence is available in the NCBI database as NP_{—}000259 (isoform 1), NP_{—}861546 (isoform 2), NP_{—}861964 (isoform 3), and NP_{—}861965 (isoform 4). Their corresponding nucleic acid sequences are NM_{—}000268.2, NM_{—}181825.1, NM_{—}181826.1, NM_{—}181827.1, respectively.

In addition, the ERM family proteins also include different post-translationally modified forms, such as phosphorylated forms.

Furthermore, the ERM family proteins include various mutant forms found, e.g., in diseased cells, such as cancers. For example, Applicants have identified several mutant forms of ezrin in cancers, the sequences of which are deposited in GenBank as AF199015 (partial human ezrin gene sequence in human epidermal carcinoma), AF190059 (mutation of ezrin gene in glioblastoma), AF189213 (a human ezrin gene mutation in cancer), AF188897 (human ezrin gene mutation in ovarian cancer), and AF188896 (mutation of human ezrin gene in brain cancer).

Other variants, homologs, isoforms, fragments, polymorphisms of the above-described proteins can be readily obtained from a sequence homology search (such as NCBI BLAST search) in public (such as GenBank, EMBL, etc.) and/or private databases. These sequences may be used to produce recombinant ERM proteins, and further used in generating ERM protein antagonists, such as antibodies (e.g., antibodies raised against the N-terminal portions of the ERM proteins).

### Exemplary ERM-Associated Diseases

The ERM family proteins have been associated with a number of disease conditions, all of which are contemplated to be treated/diagnosed by the subject methods. Some exemplary disease conditions are briefly described below.

Ezrin expression is correlated with cell motility, invasion, and cancer metastasis. It is known that ezrin is over-expressed in metastatic cancer to a greater extent than primary cancers, which in turn express higher levels than normal tissues. Ezrin levels correlate with invasive behavior and prognosis of endometrial and ovarian cancers and cancer cells. It is important that ezrin is present at low levels or absent in most normal tissues which indicates that ezrin-targeted therapies should be well tolerated.

Endometrial adenocarcinoma are the most common gynecologic cancers, and their incidence in countries like Japan has increased year by year due to the ongoing changes in life style (Japan Vital Statistics, Statistics and Information Department, Ministry of Health and Welfare, Tokyo, 1995). UEC (uterine endometroid adenocarcinoma), one of the major histologic types of endometrial adenocarcinoma, is thought to progress through a series of histologic changes from normal to hyperplasia to adenocarcinoma with the accumulation of genetic alterations in response to unopposed estrogen stimulation (Key, Mutat. Res. 333: 59-67, 1995).

Applicants have demonstrated that ezrin transcription is required for in vitro invasion and is involved in the acquisition of metastatic potential in endometrial cancer cells (Ohtani et al., Cancer Letters 147: 31-38, 1999). Applicants also examined ezrin protein expression in 20 cancerous and 33 non-cancerous tissues using immunohistochemistry and Western blot analysis (Ohtani et al., Cancer Letters 179: 79-86, 2002). The specimens included 20 uterine endometrioid adenocarcinomas (UEC), seven simple endometrial hyperplasias (sH), seven complex endometrial hyperplasias (cH), seven atypical endometrial hyperplasias (aH), and 12 samples of normal endometrium (NE). Tissues of primary (P) and metastatic (M) lesions of endometrial cancers were obtained from five patients. Ezrin was specifically expressed in UEC and its precursor lesions. Ezrin expression was significantly higher in aH (P<0.05) and UEC (P<0.001) compared with NE, sH, and cH. In addition, ezrin was significantly highly expressed in M compared with P (P<0.05). Ezrin expression was associated with neither clinical stage nor histopathologic grade of UEC. In immunohistochemistry, ezrin was localized in the membrane of metastasized cancer cells, although ezrin was mainly distributed in the cytoplasm of most cancer cells and some endometrial hyperplastic cells. On Western blot analysis, ezrin was also detected in both cytosolic and membrane fractions in aH and UEC, whereas ezrin was detected in only cytosolic fraction in sH and cH.

These data are surprising, since, given the widely-believed subcellular localization of ERM proteins (i.e., inside the plasma membrane), it would not have been expected that an ERM binding agent would bind, or inhibit the function of, the ERM proteins. Nor would one expect to detect ERM proteins with such binding agents in the various body fluids.

These data suggest that Ezrin was expressed at significantly higher levels in UEC than in NE. The specific expression of ezrin indicates that ezrin plays an active role in the development of UEC. Endometrial hyperplasias are premalignant precursors of invasive UEC. Ezrin protein expression was observed in such precursor lesions and significantly increased in aH which progresses to invasive cancer more frequently than sH and cH. Thus, ezrin protein expression may occur relatively early in endometrial tumorigenesis.

In comparison with the primary lesions, cancer cells in invasive and metastatic lesions showed stronger expression of ezrin protein. The levels of ezrin protein expression were higher in the metastatic lesions than in the matched primary lesions. The high-metastatic endometrial cancer cells, which revealed high ezrin expression at a post-transcriptional level, showed higher invasive ability and more aggressive behavior compared with the clonally maternal low-metastatic endometrial cancer cells. Thus, high expression of ezrin protein in the metastatic lesions and cancer cells with high metastatic potential is consistent with the notion that ezrin is involved in the late process of tumor progression, including invasion and metastasis.

Applicants also conducted ezrin subcellular localization studies, and demonstrated that translocation of ezrin from the cytoplasm to the areas of the membrane may occur during tumor progression and be associated with the metastatic potential. Recent evidence has demonstrated that full-length ezrin exists in a dormant state (“closed” or inactive form) in which biologically relevant binding sites are conformationally masked with an intramolecular interaction between the amino- and carboxy-terminal domains (Bretscher, Curr. Opin. Cell Biol. 11:109-116, 1999). Some signals such as phosphorylation by growth factors may disrupt this intramolecular association, allowing the conformational activation (the “open” or activated form) and formation of oligomeric surface linking structures (Bretscher, Curr. Opin. Cell Biol. 11:109-116, 1999).

### Exemplary Antagonists or Binding Agents

As used herein, the binding agents of the present invention include any compound (agent) which binds to one or more target ERM family proteins. In some embodiments, the compound inhibits the function of the ERM proteins in proliferation, invasion, and/or metastasis. Generally, such binding agents act as antagonists of the ERM protein function, and can be used as therapeutic agents for treatment of a normal or pathological condition associated with the target ERM protein, or diagnostic agents for detecting the presence and/or measuring the quantity of ERM proteins or ERM protein-containing complexes. Such binding agents may include, but are not limited to, a protein, a peptide, a small molecule (e.g. organic molecule), a peptidomimetic, an antibody (e.g., full-length, or functional fragment, derivative thereof). In one embodiment, the binding agents of the invention bind to the N-terminal part of the ERM proteins. For example, in the case of ERM family proteins, one embodiment of the ERM binding agents of the invention bind to the ERM domain/motif used for cell surface receptor interaction, and prevent the binding of ERM proteins to these cell surface receptors.

“Bind” or its various grammatical variants is used interchangeably with “interact.” It includes specific binding to a given target, such as a specific ERM family protein (e.g., ezrin). It also includes relative broad spectrum of binding to several related ERM family proteins, especially when the these proteins bound by the binding agent share high sequence homology (e.g., at least about 50% amino acid sequence identity, or at least about 60%, 70%, 80%, 90%, 95%, 97%, 99% or more identical), at least high sequence homology in the region bound by the binding agent.

“Functional” when used in the context such as “functional fragment/derivative/fusion” of a binding agent includes those fragments (e.g., less than full-length or the complete parent molecule), or derivatives, or fusions with other moieties, that substantially retain the ability to bind a target molecule bound by their parent molecules. For example, a functional fragment of an antibody, such as Fv, retains the V region of the antibody molecule, which can bind an antigen in substantially the same manner as the complete antibody does. The functional fragment/derivative/fusion of a binding agent may retain at least about 30%, 40%, 50%, 60%, 70%, 80%, 90%, or 100% of the parent molecule binding ability. In certain embodiments, the functional fragment/derivative/fusion of a binding agent may even possess a higher binding affinity to the target than the parent molecule does. Preferably, the binding agent or functional fragment/derivative/fusion thereof may inhibit at least one function of the binding partner.

There is a powerful theoretical similarity between the effect of anti-EGFR antibody (HERCEPTIN®) and the binding agents of the present invention. However, the binding agents or antagonists of the invention confer several advantages over the traditional antagonists targeting the extracellular domains of a target molecule. For example, one advantage of the ERM binding agents of the invention is that they act more proximally to signal transduction via the Rho-pathway. Many cell surface receptors (such as the CD44, EGF family (EGFR and c-erbB-2, etc.), Estrogen Receptor (ER), IL-1α receptor, and IGF family receptors), upon activation, lead to ERM protein (e.g., Ezrin) phosphorylation, such as via the Rho GTPase signaling pathway. ERM-protein phosphorylation leads to the conversion of the “closed” or dormant forms of the ERM proteins to their “open” or active forms, allowing the N-terminal part of the ERM proteins to bind the cell surface receptors, while the C-terminal part of the proteins to bind actins. Therefore, binding of the subject ERM binding agents to the N-terminal part of the activated ERM proteins (which is intercalated in the cell membrane and available to ERM binding agents administered in vivo) is not dependent on antagonizing a single, specific cell membrane receptor (such as antagonizing EGFR by Herceptin). Rather, such ERM binding agents inhibit the function of many different cancer cell membrane receptors, such as CD44, the IGF family, the EGF family receptors, Estrogen Receptor (ER), IL-1α receptor, etc. This in turn results in a greater therapeutic efficacy.

Another advantage of the ERM binding agents of the invention lies in the fact that ERM proteins (e.g., ezrin) are expressed in amounts several folds greater in cancers than in normal tissues. Therefore, it is possible to give stronger doses of the subject ERM binding agents without causing serious side effects (if any). The therapeutic index of the instant methods are relatively high.

In certain aspects, the ERM binding agents include a polypeptide which is a mutated form, a mimic or a fragment of a polypeptide that naturally binds to the ERM protein. Such polypeptides can bind to the target ERM protein and, in some embodiments, inhibit its function in, for instance, proliferation, invasion, and/or metastasis. For example, the subject ERM binding agent may include a soluble polypeptide having the amino acid sequence of EBP50 (such as NHERF and NHERF2), SAP97, palladin, L1, MRP2, L-selectin, Neutral endopeptidase 24.11 (NEP), ICAM-2, ICAM-3, RhoGDI, Db1, CD44, CD43, or the ERM binding portion thereof. Since the ERM binding region of these full-length proteins is either known or can be readily determined using art-recognized techniques (such as in vitro binding assay using various deletion fragments of the protein, etc.), such dominant negative mutated form, mimic or fragments can be readily made by the skilled person. Other variant binding sequences or peptidomimetics designed based on these peptides may also be readily obtained by using art-recognized methods, such as random mutagenesis coupled with screening for ERM binding.

In certain aspects, the binding agents of the invention can be antibodies, such as antibodies that are specifically reactive with at least one ERM family proteins. Antibodies may be polyclonal or monoclonal; intact or truncated, e.g., F(ab′)2, Fab, Fv; xenogeneic, allogeneic, syngeneic, or modified forms thereof, such as humanized or chimeric antibodies. Alternatively, these antibodies may be encoded by polynucleotides, and expressed upon transfection of such polynucleotides into the target cancer cell.

Although monoclonal antibodies (mAbs) generated from hybridoma technology have proved to be immensely useful scientific research and diagnostic tools, they have had a limited success in human therapy. Although murine antibodies have exquisite specificity for therapeutic targets, they do not always trigger the appropriate human effector's systems of complement and Fc receptors. More importantly, the major limitation in the clinical use of rodent monoclonal antibodies is an antiglobulin response during therapy. See Miller et al., Blood 62: 988-995, 1983; and Schroff et al., Cancer Res. 54: 879-885, 1985. The patient's immune system normally cuts short the therapeutic window, as murine antibodies are recognized by a human anti-mouse antibody immune response (HAMA). Similar to serum therapy where antisera used to neutralize pathogen in acute diseases and also prophylactically leads to “serum sickness”, the patient treated with rodent mAbs in multiple doses invariably raises an immune response to the mAbs, manifesting similar symptoms to serum sickness. This response can occur within two weeks of the initiation of treatment and precludes long-term therapy. Thus efforts have been made to raise human mAbs against therapeutic targets through immortalization of human antibody-producing cells.

To produce therapeutic antibodies with high binding affinity, reduced immunogenicity (HAMA response), increased half-life in the human body and adequate recruitment of effectors functions (i.e., the ability to summon the body's own natural defense), the techniques of monoclonal antibody production and recombinant DNA technology are combined to overcome the problem associated with rodent monoclonal antibodies. Besides direct generation of fully human antibody, another popular approach is to humanize rodent monoclonal antibody. See, for example, Queen et al., Proc. Natl. Acad. Sci. USA 86: 10029-10033, 1989), and U.S. Pat. No. 5,693,762.

US20040067532A1 (incorporated herein by reference) describes compositions, methods, and kits for efficiently generating and screening humanized antibody with high affinity against a specific antigen. According to that method, a library of humanized antibody is generated by mutagenizing a chimeric antibody template that combines human antibody framework and antigen binding sites of a non-human antibody. Alternatively, the library of humanized antibody is generated by grafting essential antigen-recognition segment(s) of the non-human antibody into the corresponding position(s) of each member of a human antibody library. This library of humanized antibody is then screened for high affinity binding toward a specific antigen in vivo in organism such as yeast or in vitro using techniques such as ribosome display or mRNA display. The specific antigen used in the screening can be the one against which the non-human antibody is originally elicited, or an antigen with similar structural features or biological function. In addition, the library of humanized antibody may be used in screening for high affinity antibody against an antigen that is structurally and/or functionally different from the antigen against which the non-human antibody is originally elicited. These selection processes can be performed to select antibody having higher affinity in antigen binding but lower immunogenecity than rodent monoclonal antibody. The overall process can be efficiently performed in a high throughput and automated manner, thus mimicking the natural process of antibody affinity maturation.

While not wishing to be bound by any particular theory, these antibodies or fragments thereof may bind the ERM proteins on the surface of the cells (such as the intracellular surface of the cell), or ERM proteins accessible by extracellular binding agents, and antagonize ERM protein function in proliferation, invasion, and/or metastasis. Alternatively, since ERM proteins are normally thought to be intracellular proteins not accessible to large extracellular molecules, immune system of the host may recognize and eliminate such “foreign” antibody-engaged ERM structures on proliferative cells, through, for example, natural killer cells (NK cells). Thus by providing a subject ERM protein binding agent complex (e.g., antibody-engaged ERM complex), the host immune system may be stimulated or immunized against such ERM-associated proliferative conditions.

For example, by using immunogens derived from a target ERM protein, or from several ERM proteins, anti-protein/anti-peptide antisera or monoclonal antibodies can be made by standard protocols (see, for example, Antibodies: A Laboratory Manual ed. by Harlow and Lane (Cold Spring Harbor Press: 1988)). A mammal, such as a mouse, a hamster or rabbit can be immunized with an immunogenic form of the peptide (e.g., a polypeptide or an antigenic fragment which is capable of eliciting an antibody response, or a fusion protein). Techniques for conferring immunogenicity on a protein or peptide include conjugation to carriers or other techniques well known in the art. A full-length or an immunogenic portion of an ERM protein can be administered in the presence of adjuvant. The progress of immunization can be monitored by detection of antibody titers in plasma or serum. Standard ELISA or other immunoassays can be used with the immunogen as antigen to assess the levels of antibodies.

Following immunization of an animal with an antigenic preparation of an ERM protein, antisera can be obtained and, if desired, polyclonal antibodies can be isolated from the serum. In certain embodiments, polyclonal antibodies (antisera, affinity purified polyclonal antibodies, etc.) may be preferred, since relatively little is known about the metabolism of ERM proteins such as ezrin, and thus the use of polyclonal antibodies may in some cases detect certain fragments of ERM proteins that may not be bound by the usual monoclonal antibodies.

To produce monoclonal antibodies, antibody-producing cells (lymphocytes) can be harvested from an immunized animal and fused by standard somatic cell fusion procedures with immortalizing cells such as myeloma cells to yield hybridoma cells. Such techniques are well known in the art, and include, for example, the hybridoma technique (originally developed by Kohler and Milstein, Nature 256: 495-497, 1975), the human B cell hybridoma technique (Kozbar et al., Immunology Today 4: 72, 1983), and the EBV-hybridoma technique to produce human monoclonal antibodies (Cole et al., Monoclonal Antibodies and Cancer Therapy, Alan R. Liss, Inc. pp. 77-96, 1985). Hybridoma cells can be screened immunochemically for production of antibodies specifically reactive with an ERM protein, and monoclonal antibodies isolated from a culture comprising such hybridoma cells.

The term antibody as used herein is intended to include fragments thereof which are also specifically reactive with an target protein. Antibodies can be fragmented using conventional techniques and the fragments screened for utility in the same manner as described above for whole antibodies. For example, F(ab)₂ fragments can be generated by treating antibody with pepsin. The resulting F(ab)₂ fragment can be treated to reduce disulfide bridges to produce Fab fragments. An antibody of the present invention is further intended to include bispecific, single-chain, and chimeric and humanized molecules having affinity for a target protein conferred by at least one CDR region of the antibody. Techniques for the production of single chain antibodies (U.S. Pat. No. 4,946,778) can also be adapted to produce single chain antibodies. Also, transgenic mice or other organisms including other mammalian species, may be used to express humanized antibodies. In certain embodiments, such as in the diagnosis methods, the antibodies may further comprise a label attached thereto, and are thus able to be detected (e.g., the label can be a radioisotope, fluorescent compound, enzyme or enzyme co-factor).

In certain specific embodiments, an antibody of the invention is a monoclonal antibody, and in certain embodiments the invention makes available methods for generating novel antibodies. For example, a method for generating a monoclonal antibody that binds specifically to a target protein may comprise administering to a mouse an amount of an immunogenic composition comprising the target protein effective to stimulate a detectable immune response, obtaining antibody-producing cells (e.g., cells from the spleen) from the mouse and fusing the antibody-producing cells with myeloma cells to obtain antibody-producing hybridomas, and testing the antibody-producing hybridomas to identify a hybridoma that produces a monoclonal antibody that binds specifically to the target protein. Once obtained, a hybridoma can be propagated in a cell culture, optionally in culture conditions where the hybridoma-derived cells produce the monoclonal antibody that binds specifically to the target protein. The monoclonal antibody may be purified from the cell culture.

Such techniques can be used to generate antibodies using wild-type or altered ERM proteins, such as those encoded by the polynucleotide sequences of GenBank Accession Nos. AF199015, AF190059, AF189213, AF188897, and AF188896.

In addition, the techniques used to screen antibodies in order to identify a desirable antibody may influence the properties of the antibody obtained. For example, an antibody to be used for certain therapeutic purposes will preferably be able to target an antigen on a particular cell type, as opposed to antigen in solution. Accordingly, to obtain antibodies of this type, it may be desirable to screen for antibodies that bind to cells that express the antigen of interest (e.g., by fluorescence activated cell sorting), or at least confirm that the antibody can bind to ERM proteins (especially on cell surface). A variety of different techniques are available for testing antibody:antigen interactions to identify particularly desirable antibodies. Such techniques include ELISAs, surface plasmon resonance binding assays (e.g. the BIAcore binding assay, Bia-core AB, Uppsala, Sweden), sandwich assays (e.g. the paramagnetic bead system of IGEN International, Inc., Gaithersburg, Md.), Western blots, immunoprecipitation assays and immunohistochemistry.

In certain aspects, the binding agents of the present invention include a small molecule or a peptidomimetic. Examples of small molecules include, but are not limited to, small peptides or peptide-like molecules (e.g., a peptidomimetic). As used herein, the term “peptidomimetic” includes chemically modified peptides and peptide-like molecules that contain non-naturally occurring amino acids, peptoids, and the like. Peptidomimetics provide various advantages over a peptide, including enhanced stability when administered to a subject. Methods for identifying a peptidomimetic are well known in the art and include the screening of databases that contain libraries of potential peptidomimetics. For example, the Cambridge Structural Database contains a collection of greater than 300,000 compounds that have known crystal structures (Allen et al., Acta Crystallogr. Section B 35: 2331, 1979). Where no crystal structure of a target molecule is available, a structure can be generated using, for example, the program CONCORD (Rusinko et al., J. Chem. Inf. Comput. Sci. 29: 251, 1989). Another database, the Available Chemicals Directory (Molecular Design Limited, Informations Systems; San Leandro Calif.), contains about 100,000 compounds that are commercially available and also can be searched to identify potential peptidomimetics of CCL21 or a chemokine receptor.

As described herein, small molecule compounds may encompass numerous chemical classes, although typically they are organic molecules, preferably small organic compounds having a molecular weight of more than 50, and less than about 5,000 Da, or less than 4,000 Da, or less than 3,000 Da, or less than 2,500 daltons. Candidate agents comprise functional groups necessary for structural interaction with proteins, particularly hydrogen bonding, and typically include at least an amine, carbonyl, hydroxyl, sulfhydryl or carboxyl group. Candidate small molecule compounds can be obtained from a wide variety of sources including libraries of synthetic or natural compounds. For example, numerous means are available for random and directed synthesis of a wide variety of organic compounds and biomolecules, including expression of randomized oligonucleotides. Alternatively, libraries of natural compounds in the form of bacterial, fungal, plant, and animal extracts are available or readily produced. Additionally, natural or synthetically produced libraries and compounds can be modified through conventional chemical, physical, and biochemical means. Known pharmacological agents may be subjected to directed or random chemical modifications, such as acylation, alkylation, esterification, and amidification, to produce structural analogs.

The present invention also contemplates anti-tumor therapeutic agents obtainable from the screening methods described below.

### Binding Agent Screening Assays

There are numerous approaches to screening for additional subject binding agents. For example, high-throughput screening of compounds or molecules can be carried out to first identify agents or drugs that bind the target protein (e.g., an ERM protein). New binding agents (e.g., antibodies) against a particular domain of a target protein (such as one or more specific regions of the N-terminal domain of the ERM proteins used to interact with cell surface receptors) may be raised. Such target proteins may be normal or wild-type, or can contain various post-translational modifications such as phosphorylation, or can be mutated or truncated versions found in disease cells (e.g., cancer cells). The binding agents of the invention may specifically recognize one of these forms, but not other forms; or could recognize a common epitope present in all these forms, depending on specific needs. All such binding agents may be obtained using the screening methods of the invention.

Thus the invention also provides methods to screen for binding agents that specifically or generically bind to one or more target ERM or perimembrane proteins, and binding agents identified using the subject screening methods.

Once such agents or drugs are identified, they may be further tested in one or more biological assays to determine if they inhibit at least one ERM-associated function, such as proliferation.

Any ERM binding agents may be suitable for diagnostic use, especially when the binding reaction to detect and/or quantitate ERM proteins is carried out in vitro. Some or all of these binding agents may also be suitable for use as therapeutic agents to treat and/or prevent an ERM-associated condition in vivo.

For example, any agents that are identified as ERM-binding agent may be further assessed for their ability to inhibit cancer cell invasion in a Matrigel assay, which was used to demonstrate that anti-ezrin monoclonal antibody can inhibit cancer cell invasion. Alternatively, the ERM-binding agent may be tested in an animal model for their ability to inhibit cancer metastasis in vivo. For instance, immuno-compromised animals (e.g., nude mice) may be implanted subcutaneously or intra-peritoneally with a metastatic cancer cell line (e.g., tumor xenograph implant into nude mice), and the ERM-binding agent is then administered to the animal to determine if it significantly inhibits cancer growth at the implant site (the primary site), and/or significantly reduces metastasis to a distant organ (such as lung).

Candidate binding agents to be assessed for their ability to bind a target protein, and/or ability to inhibit a function of the target protein (e.g., inhibit ERM-associated proliferation, invasion or metastasis) can be any chemical in nature (e.g., element, molecule, compound, drug), either made synthetically, or made by recombinant techniques or isolated from a natural source, or a combination thereof. For example, test agents can be peptides, polypeptides, peptoids, sugars, hormones, or nucleic acid molecules. In addition, test agents can be small molecules or molecules of greater complexity made by combinatorial chemistry, for example, and compiled into libraries. These libraries can comprise, for example, alcohols, alkyl halides, amines, amides, esters, aldehydes, ethers and other classes of organic compounds. Test agents can also be natural or genetically engineered products isolated from lysates or growth media of cells—bacterial, animal or plant—or can be the cell lysates or growth media themselves. Presentation of test compounds to the test system can be in either an isolated form or as mixtures of compounds, especially in initial screening steps.

In one embodiment of an assay to identify a test compound that binds to a target protein, the target protein may be immobilized on a solid support. The immobilized protein is then contacted with either a labeled test compound (e.g., direct binding assay), or a labeled known target protein-binding agent plus a test compound or a group of test compounds (e.g., a competitive assay). The amount of labeled is then determined. The amount of label is proportional (in the direct assay) or inversely proportional (in the competitive assay) to the ability of the test compound to bind the target protein.

The label used can be, for example, a radioactive isotope, or a fluorescent or colormetric label. The solid support can be any suitable solid phase or matrix, such as resin in a packed column, suspended beads in a solution, the wall of a plate or other suitable surface (e.g., a well of a microtiter plate), BIOCORE binding surface, column pore glass (CPG) or a pin that can be submerged into a solution, such as in a well. Linkage of the target protein to the solid support can be either direct or through one or more linker molecules.

In one embodiment, an isolated or purified target protein can be immobilized on a suitable affinity matrix by standard techniques, such as chemical cross-linking, or via an antibody raised against the isolated or purified protein, and bound to a solid support. The matrix can be packed in a column or other suitable container and is contacted with one or more compounds (e.g., a mixture) to be tested under conditions suitable for binding of the compound to the protein. For example, a solution containing compounds can be made to flow through the matrix. The matrix can be washed with a suitable wash buffer to remove unbound compounds and non-specifically bound compounds. Compounds which remain bound can be released by a suitable elution buffer. For example, a change in the ionic strength or pH of the elution buffer can lead to a release of compounds. Alternatively, the elution buffer can comprise a release component or components designed to disrupt binding of compounds (e.g., one or more ligands or receptors, as appropriate, or analogs thereof which can disrupt binding or competitively inhibit binding of test compound to the protein).

Fusion proteins comprising all of, or a portion of, a target protein linked to a second moiety not occurring in the target protein as found in nature can also be prepared for use in another embodiment of the method. Suitable fusion proteins for this purpose include those in which the second moiety comprises an affinity ligand (e.g., an enzyme, antigen, epitope). The fusion proteins can be produced by inserting the target ERM protein or a portion thereof into a suitable expression vector which encodes an affinity ligand. The expression vector can be introduced into a suitable host cell for expression. Host cells are disrupted and the cell material, containing fusion protein, can be bound to a suitable affinity matrix by contacting the cell material with an affinity matrix under conditions sufficient for binding of the affinity ligand portion of the fusion protein to the affinity matrix.

Thus in one aspect of this embodiment, a fusion protein can be immobilized on a suitable affinity matrix under conditions sufficient to bind the affinity ligand portion of the fusion protein to the matrix, and is contacted with one or more compounds (e.g., a mixture) to be tested, under conditions suitable for binding of compounds to the receptor or ligand protein portion of the bound fusion protein. Next, the affinity matrix with bound fusion protein can be washed with a suitable wash buffer to remove unbound compounds and non-specifically bound compounds without significantly disrupting binding of specifically bound compounds. Compounds which remain bound can be released by contacting the affinity matrix having fusion protein bound thereto with a suitable elution buffer (a compound elution buffer). In this aspect, compound elution buffer can be formulated to permit retention of the fusion protein by the affinity matrix, but can be formulated to interfere with binding of the compound(s) tested to the receptor or ligand protein portion of the fusion protein. For example, a change in the ionic strength or pH of the elution buffer can lead to release of compounds, or the elution buffer can comprise a release component or components designed to disrupt binding of compounds to the receptor or ligand protein portion of the fusion protein (e.g., one or more ligands or receptors or analogs thereof which can disrupt binding of compounds to the receptor or ligand protein portion of the fusion protein). Immobilization can be performed prior to, simultaneous with, or after contacting the fusion protein with compound, as appropriate. Various permutations of the method are possible, depending upon factors such as the compounds tested, the affinity matrix selected, and elution buffer formulation. For example, after the wash step, fusion protein with compound bound thereto can be eluted from the affinity matrix with a suitable elution buffer (a matrix elution buffer). Where the fusion protein comprises a cleavable linker, such as a thrombin cleavage site, cleavage from the affinity ligand can release a portion of the fusion with compound bound thereto. Bound compound can then be released from the fusion protein or its cleavage product by an appropriate method, such as extraction.

In some cases, one or more compounds can be tested simultaneously. Where a mixture of compounds is tested, the compounds selected by the foregoing processes can be separated (as appropriate) and identified by suitable methods (e.g., PCR, sequencing, chromatography). Large combinatorial libraries of compounds (e.g., organic compounds, peptides, nucleic acids) produced by combinatorial chemical synthesis or other methods can be tested (see e.g., Ohlmeyer, M. H. J. et al., Proc. Natl. Acad. Sci. USA 90: 10922-10926, 1993; and DeWitt, S. H. et al., Proc. Natl. Acad. Sci. USA 90: 6909-6913, 1993, relating to tagged compounds. See also, Rutter, W. J. et al., U.S. Pat. No. 5,010,175; Huebner, V. D. et al., U.S. Pat. No. 5,182,366; and Geysen, H. M., U.S. Pat. No. 4,833,092). Where compounds selected from a combinatorial library by the present method carry unique tags, identification of individual compounds by chromatographic methods is possible. Where compounds do not carry tags, chromatographic separation, followed by mass spectrophotometry to ascertain structure, can be used to identify individual compounds selected by the method, for example.

The instant invention also provides a method to identify agents that inhibit one or more ERM family proteins. In this embodiment, a plurality of agents are first identified as antagonists to at least one target protein using any of the above described methods. Then these antagonists may be further tested, using any of the above methods, for their ability to inhibit a second (or third, etc.) family protein. Alternatively, the method can be carried out in an array format, in that a number of related target proteins may be tested simultaneously against one or more individual test compound(s) (such as immobilizing the target ERMs on an array, and contacting the array with one or more test compounds). If each test compounds is labeled by a unique identifiable tag, the types and/or amounts of each tagged compound bound to each target proteins on the array can be simultaneously determined.

Inhibitors in the present invention can also be designed by using molecular modeling. A computer model of a target protein or a close homolog thereof may be used to identify any compounds that might bind the target protein in the ligand binding sites. Alternatively, antagonistic compounds mimicking the natural ligands of these targets might be designed in silica. Alternatively, the nature of the inhibitory sequence can be determined by calculation, based on knowledge of a receptor or binding pocket. Other calculational strategies will be known to those skilled in the art. Calculations such as these can be useful for directing the synthesis of inhibitors of the present invention in a time- and material-efficient manner, before actual synthesis and screening techniques begin.

Other methods that can be adapted for screening the binding agents of the present invention are well known in the art, independent of the use of computer modeling. The use of peptide libraries is one way of screening large numbers of polypeptides at once. In one screening assay, the candidate peptides are displayed on the surface of a cell or viral particle, and the ability of particular cells or viral particles to bind a target ERM protein is detected in a “panning assay.” For instance, the gene library can be cloned into the gene for a surface membrane protein of a bacterial cell, and the resulting chimeric polypeptide detected by panning (Ladner et al., WO 88/06630; Fuchs et al. (1991) Bio/Technology 9:1370-1371; and Goward et al. (1992) TIBS 18:136-140).

In an alternate embodiment, the peptide library to be screened is expressed as chimeric polypeptides on the surface of a viral particle. For instance, in the filamentous phage system, foreign peptide sequences can be expressed on the surface of infectious phage, thereby conferring two significant benefits. First, since these phage can be applied to affinity matrices at very high concentrations, a large number of phage can be screened at one time. Second, since each infectious phage displays the combinatorial gene product on its surface, if a particular phage is recovered from an affinity matrix in low yield, the phage can be amplified by another round of infection. The group of almost identical E. coli filamentous phages M13, fd, and fl are most often used in phage display libraries, as either of the phage gIII or gVIII coat proteins can be used to generate chimeric polypeptides without disrupting the ultimate packaging of the viral particle (Ladner et al., WO 90/02809; Garrard et al., WO 92/09690; Marks et al., J. Biol. Chem. 267: 16007-16010, 1992; Griffiths et al., EMBO J 12: 725-734, 1993; Clackson et al., Nature 352: 624-628, 1991, and Barbas et al., PNAS 89: 4457-4461, 1992).

The field of combinatorial peptide libraries has been reviewed (Gallop et al. J. Med. Chem. 37: 1233-1251, 1994), and additional techniques are known in the art (Gustin, K Virology 193: 653-660, 1993; Goeddel et al. U.S. Pat. No. 5,223,408; Markland et al. PCT publication WO92/15679; Bass et al. Proteins: Structure, Function and Genetics 8: 309-314, 1990; Cunningham, B. C. Science 247: 1461-1465, 1990; Lowman, H. B. Biochemistry 30: 10832-10838, 1991; Fowlkes et al. U.S. Pat. No. 5,789,184; Houghton, Proc. Natl. Acad. Sci. U.S.A. 82: 5131-5135, 1985) for generating and screening peptide libraries.

U.S. Pat. No. 6,420,110 (incorporated herein by reference) discloses a method for isolating biologically active peptides. Using the techniques disclosed therein, a polypeptide ERM protein inhibitor of the present invention may be developed, which interacts with a chosen ERM protein, and inhibits the function thereof. The inhibition can be readily tested in many suitable in vitro or in vivo models, such as those described herein.

In a representative example, this method is utilized to identify polypeptide ERM protein antagonists which have antagonistic activity with respect to one or more types of cells expressing at least one ERM protein. One of skill in the art will readily be able to modify the procedures outlined below to find polypeptides with any desired activity. In the example, in the display mode, the chimeric polypeptide library can be panned with the target cells or immobilized target protein in order to enrich for polypeptides which bind to that cell or receptor. At that stage, the polypeptide library can also be panned against one or more control cell lines (that does not express any of the target proteins) in order to remove polypeptides which bind the control cells. In this manner, the polypeptide library which is then tested in the secretion mode can be enriched for polypeptides which selectively bind target cells (relative to the control cells). Thus, for example, the display mode can produce a polypeptide library enriched for polypeptides which preferentially bind ERM-expressing tumor cells relative to normal cells, or any other differential binding characteristic.

In the secretion mode, the polypeptides are tested for antiproliferative and/or invasive activity against the target cell, using any of a number of techniques known in the art. For instance, BrdU or other nucleotide uptake can be measured as an indicator of proliferation. Matrigel invasion assay may be used to test invasiveness. Animal models (e.g., tumor xenograph in nude mice) may be used to test metastasis, etc. Other suitable functional test for specific ERM proteins are well-known in the art. Furthermore, the secretion mode can include negative controls in order to select for polypeptides with specific biological activity (e.g., antiproliferative/anti-invasiveness/anti-metastatic activity), rather than non-specific effects such as general toxicity.

### Exemplary ERM-Interacting Proteins

As described above, any ERM binding proteins must contain a domain, motif or moiety (e.g., a peptide fragment) that binds ERM. Such ERM-binding peptides may interfere with ERM protein function, and thus serve as candidate molecules for testing their ability as ERM antagonists. Some of the known ERM binding proteins are listed below. Other ERM binding proteins may be readily identified using any art-recognized techniques for identifying protein-protein interaction, such as various kinds of interaction-trap assays (e.g., yeast two-hybrid assays), phage display, etc.

Bonilha and Rodriguez-Boulan (Invest. Ophthal. Vis. Sci. 42: 3274-3282, 2001) identified EBP50 and SAP97 as binding partners for ezrin, an actin-binding protein crucial for morphogenesis of apical microvilli and basolateral in foldings in retinal pigment epithelial (RPE) cells. Immunofluorescence microscopy detected a polarized distribution of EBP50 at apical microvilli and of SAP97 at the basolateral surface of RPE cells, which overlapped with ezrin.

By two-hybrid analysis, affinity precipitation, and mutation analysis, Mykkanen et al. (Molec. Biol. Cell 12: 3060-3073, 2001) determined that the alpha-helical region of ezrin interacted with the C-terminal Ig domains of the microfilament-associated protein palladin. The palladin-binding site was masked in dormant wild-type ezrin. By double staining of ezrin and palladin in several cell lines, Mykkanen et aL (supra) found that the subcellular localization of ezrin differed between epithelia and smooth muscle cells. In epithelial cells, such as HeLa, ezrin localized at the cortical actin skeleton and demonstrated little overlap with palladin. However, in intestinal smooth muscle cells, ezrin demonstrated a filamentous staining pattern and partial co-localization with palladin.

Cheng et al. (J Neurosci. 25(2): 395-403, 2005) identified two regions on the neural cell adhesion molecule L1 as ERM-binding sites—the RSLE region and a novel juxtamembrane ERM-binding region.

Morales et al. (Proc Natl Acad Sci USA. 101(51): 17705-10, 2004) indicated that the ERM-binding phosphoprotein 50/Na⁺/H⁺ exchanger regulatory factor 1 (EBP50/NHERF1) binds the N-terminal domain of ERM proteins. Terawaki et al. (Acta Crystallogr D Biol Crystallogr. 59(Pt 1): 177-9, 2003) previously reported the crystal structure of the complexes between the radixin FERM domain and the C-terminal regions of NHERF and NHERF2.

In vitro binding studies showed that radixin associates directly with the carboxy-terminal cytoplasmic domain of human MRP2 (multidrug resistance protein-2). NF2 tumor suppressor protein and radixin also interacts with the carboxy-terminal domain of layilin, a cell surface hyaluronan receptor (Bono et al., Exp. Cell Res. May 20, 2005, epublication).

Ivetic et al. (J. Biol Chem. 279(32): 33263-72, 2004) have not only identified ezrin and moesin as binding partners of the 17-amino acid L-selectin tail, but also two basic amino acid residues within that L-selectin tail as being required for binding to ezrin-radixinmoesin (ERM) proteins: arginine 357 and lysine 362. Serrador et al. (Eur J Immunol. 32(6): 1560-6, 2002) also found that a juxta-membrane amino acid sequence of P-selectin glycoprotein ligand-1 (PSGL-1) is involved in moesin binding.

Iwase et al. (J Biol Chem. 279(12): 11898-905, 2004) found that the cytoplasmic domain of Neutral endopeptidase 24.11 (NEP) contains a positively charged amino acid cluster, which binds the N terminal fragment of ezrin/radixin/moesin (ERM) proteins. Binding of ERM proteins to NEP results in decreased binding of ERM proteins to the hyaluronan receptor CD44, a main binding partner of ERM proteins. Moreover, cells expressing wild-type NEP demonstrate decreased adhesion to hyaluronic acid and cell migration.

Hamada et al. (EMBO J. 22(3): 502-14, 2003) reported the crystal structure of the radixin FERM (4.1 and ERM) domain complexed with the ICAM-2 cytoplasmic peptide. The non-polar region of the ICAM-2 peptide contains the RxxTYxVxxA sequence motif to form a beta-strand followed by a short 3(10)-helix. It binds the groove of the phosphotyrosine-binding (PTB)-like subdomain C mediated by a beta-beta association and several side-chain interactions. The binding mode of the ICAM-2 peptide to the FERM domain is distinct from that of the NPxY motif-containing peptide binding to the canonical PTB domain. Mutation analyses based on the crystal structure reveal the determinant elements of recognition and provide the first insights into the physical link between adhesion molecules and ERM proteins.

Serrador et al. (J Biol Chem. 277(12):10400-9, 2002) also reported a phosphatidylinositol 4,5-bisphosphate-induced association between ICAM-3 and the amino-terminal domain of ERM proteins, and the role of specific serine residues (Ser487 and Ser489, possibly also Ser496) within the cytoplasmic region of ICAM-3 for its ERM-directed positioning at the trailing edge of motile lymphocytes.

Hamada et al. (Acta Crystallogr D Biol Crystallogr. 57(Pt 6): 889-90, 2001) reported the crystal structure of RhoGDI complexed with the FERM domain of radixin. Takahashi et al. (Oncogene 16(25): 3279-84, 1998) showed that the N-terminal region of radixin furthermore interacts with Dbl, a stimulatory GDP/GTP exchange protein of the Rho family members. This interaction does not affect the Dbl activity to stimulate the GDP/GTP exchange reaction of RhoA, a member of the Rho subfamily. Dbl does not interact with radixin which is precomplexed with Rho GDI, and Rho GDI displaces Dbl from radixin.

Yonemura et al. (J Cell Biol. 140(4): 885-95, 1998) reported that ERM proteins bind to a positively charged amino acid cluster in the juxta-membrane cytoplasmic domain of CD44, CD43, and ICAM-2.

### Methods of Treatment

In certain embodiments, the present invention provides methods of treating an individual suffering from a proliferative condition, such as cancer, through administering to the individual a therapeutically effective amount of an ERM binding agent as described above.

In other embodiments, the invention provides methods of preventing or delaying the onset, and/or retarding the progression of the proliferative condition (e.g., cancer) in an individual through administering to the individual a therapeutically effective amount of an ERM binding agent. These methods are particularly aimed at therapeutic and prophylactic treatments of animals, and more particularly, humans. In these embodiments, the ERM binding agent binds an ERM protein and inhibits at least one function of the ERM protein.

In certain embodiments of such methods, one or more ERM binding agents can be administered, together (simultaneously) or at different times (sequentially). In addition, ERM binding agents can be administered with one or more other compounds for treating the proliferative condition (e.g. cancer). The two or more compounds may be administered simultaneously or sequentially.

Methods of the present invention can be used to treat a variety of proliferative conditions, including cancer and (benign) proliferative disorders.

The cancers that can be treated using the subject method include, but are not limited to: ovarian cancer, endometrial cancer, breast cancer, glioblastoma, schwannoma, meningioma, malignant mesothelioma, neurofibromatosis, colon cancer, oral cancer, or a cancer selected from the group consisting of: lung cancer, prostate cancer, pancreatic cancer, leukemia, liver cancer, stomach cancer, uterine cancer, testicular cancer, brain cancer, non-hodgkin's lymphoma, hodgkin's lymphoma, Ewing's sarcoma, osteosarcoma, neuroblastoma, rhabdomyosarcoma, melanoma, and brain cancer.

The cancer may be invasive and/or metastatic.

The benign proliferative disorders that can be treated using the subject method include, but are not limited to: tuberosclerosis, psoriasis, endometriosis, or polyps (such as colon polyps).

### Pharmaceutical Compositions

In certain embodiments, the ERM binding agents of the present invention are formulated as a pharmaceutical composition with a pharmaceutically acceptable carrier or salt. The term “pharmaceutically acceptable salts” refers to physiologically and pharmaceutically acceptable salts of the compounds of the invention, e.g., salts that retain the desired biological activity of the parent compound and do not impart undesired toxicological effects thereto.

The ERM binding agents, when used as therapeutic agents, can be administered alone or as a component of a pharmaceutical formulation (composition). The ERM binding agents may be formulated for administration in any convenient way for use in human or veterinary medicine. In certain embodiments, the ERM binding agents included in the pharmaceutical preparation may themselves be active, or may be prodrugs. The term “prodrug” refers to compounds which, under physiological conditions, are converted into therapeutically active agents (such as a binding agent that is normally inhibited before administration, but the inhibition is removed by enzymatic cleavage or pH change, etc., when the binding agent is delivered in vivo to the individual).

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Pharmaceutical compositions of the ERM binding agents include those suitable for oral/nasal, topical, parenteral and/or intravaginal administration. The pharmaceutical compositions may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect.

Methods of preparing these pharmaceutical compositions or compositions include combining an ERM binding agent and a carrier, and optionally, one or more accessory ingredients. In general, the pharmaceutical compositions can be prepared with a liquid carrier, or a finely divided solid carrier, or both, and then, if necessary, shaping the product.

Pharmaceutical compositions for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of an artemisinin-related compound as an active ingredient. An artemisinin-related compound may also be administered as a bolus, electuary or paste.

In solid dosage forms for oral administration (capsules, tablets, pills, dragees, powders, granules, and the like), one or more ERM binding agents of the present invention may be mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose, and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

Liquid dosage forms for oral administration include pharmaceutically acceptable emulsions, microemulsions, solutions, suspensions, syrups, and elixirs. In addition to the active ingredient, the liquid dosage forms may contain inert diluents commonly used in the art, such as water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor, and sesame oils), glycerol, tetrahydrofuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, coloring, perfuming, and preservative agents.

Suspensions, in addition to the active compounds, may contain suspending agents such as ethoxylated isostearyl alcohols, polyoxyethylene sorbitol, and sorbitan esters, microcrystalline cellulose, aluminum metahydroxide, bentonite, agar-agar and tragacanth, and mixtures thereof.

In particular, therapeutic agents or pharmaceutical compositions can be topically, either to skin or to mucosal membranes, such as those of the cervix and vagina. The topical pharmaceutical compositions may further include one or more of the wide variety of agents known to be effective as skin or stratum corneum penetration enhancers. Examples of these are 2-pyrrolidone, N-methyl-2-pyrrolidone, dimethylacetamide, dimethylformamide, propylene glycol, methyl or isopropyl alcohol, dimethyl sulfoxide, and azone. Additional agents may further be included to make the formulation cosmetically acceptable. Examples of these are fats, waxes, oils, dyes, fragrances, preservatives, stabilizers, and surface active agents. Keratolytic agents such as those known in the art may also be included. Examples are salicylic acid and sulfur.

Dosage forms for the topical or transdermal administration include powders, sprays, ointments, pastes, creams, lotions, gels, solutions, patches, and inhalants. The active compound may be mixed under sterile conditions with a pharmaceutically acceptable carrier, and with any preservatives, buffers, or propellants which may be required. The ointments, pastes, creams and gels may contain, in addition to an artemisinin-related compound, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an ERM binding agents, excipients such as lactose, talc, silicic acid, aluminum hydroxide, calcium silicates, and polyamide powder, or mixtures of these substances. Sprays can additionally contain customary propellants, such as chlorofluorohydrocarbons and volatile unsubstituted hydrocarbons, such as butane and propane.

Pharmaceutical compositions suitable for parenteral administration may comprise one or more ERM binding agents in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents. Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants, such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption, such as aluminum monostearate and gelatin.

Injectable depot forms are made by forming microencapsule matrices of one or more anti-tumor therapeutic agents in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable pharmaceutical compositions are also prepared by entrapping the drug in liposomes or microemulsions which are compatible with body tissue.

Pharmaceutical compositions for intravaginal administration may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound. Optionally, such pharmaceutical compositions suitable for vaginal administration also include pessaries, tampons, creams, gels, pastes, foams or spray pharmaceutical compositions containing such carriers as are known in the art to be appropriate.

### Methods of Administration

In certain embodiments, the subject treatment methods of the invention can be used alone. Alternatively, the subject treatment methods may be used in combination with other conventional anti-proliferative therapeutic approaches directed to treatment or prevention of proliferative disorders (e g., tumors). For example, such methods can be used in prophylactic cancer prevention, prevention of cancer recurrence and metastases after surgery, and as an adjunct to other conventional cancer therapies. The present invention recognizes that the effectiveness of conventional cancer therapies (e.g., chemotherapy, radiation therapy, phototherapy, immunotherapy, and surgery) can be enhanced through the use of an ERM binding agent which inhibits ERM protein function.

A wide variety of conventional compounds have been shown to have anti-neoplastic activities. These compounds have been used as pharmaceutical agents in chemotherapy to shrink solid tumors, prevent metastases and further growth, or decrease the number of malignant cells in leukemic or bone marrow malignancies. Although chemotherapy has been effective in treating various types of malignancies, many anti-neoplastic compounds induce undesirable side effects. It has been shown that when two or more different treatments are combined, the treatments may work synergistically and allow reduction of dosage of each of the treatments, thereby reducing the detrimental side effects exerted by each compound at higher dosages. In other instances, malignancies that are refractory to a treatment may respond to a combination therapy of two or more different treatments.

When a ERM binding agent of the present invention is administered in combination with another conventional anti-neoplastic agent, either concomitantly or sequentially, such therapeutic agent may enhance the therapeutic effect of the anti-neoplastic agent or overcome cellular resistance to such anti-neoplastic agent. This may allow decrease of dosage of an anti-neoplastic agent, thereby reducing the undesirable side effects, or restores the effectiveness of an anti-neoplastic agent in resistant cells.

Pharmaceutical compounds that may be used for such combination chemotherapy include, merely to illustrate: aminoglutethimide, amsacrine, anastrozole, asparaginase, bcg, bicalutamide, bleomycin, buserelin, busulfan, campothecin, capecitabine, carboplatin, carmustine, chlorambucil, cisplatin, cladribine, clodronate, colchicine, cyclophosphamide, cyproterone, cytarabine, dacarbazine, dactinomycin, daunorubicin, dienestrol, diethylstilbestrol, docetaxel, doxorubicin, epirubicin, estradiol, estramustine, etoposide, exemestane, filgrastim, fludarabine, fludrocortisone, fluorouracil, fluoxymesterone, flutamide, gemcitabine, genistein, goserelin, hydroxyurea, idarubicin, ifosfamide, imatinib, interferon, irinotecan, ironotecan, letrozole, leucovorin, leuprolide, levamisole, lomustine, mechlorethamine, medroxyprogesterone, megestrol, melphalan, mercaptopurine, mesna, methotrexate, mitomycin, mitotane, mitoxantrone, nilutamide, nocodazole, octreotide, oxaliplatin, paclitaxel, pamidronate, pentostatin, plicamycin, porfimer, procarbazine, raltitrexed, rituximab, streptozocin, suramin, tamoxifen, temozolomide, teniposide, testosterone, thioguanine, thiotepa, titanocene dichloride, topotecan, trastuzumab, tretinoin, vinblastine, vincristine, vindesine, vinorelbine, and HERCEPIIN® and other antibody therapeutics.

These chemotherapeutic compounds may be categorized by their mechanism of action into, for example, following groups: anti-metabolites/anti-cancer agents, such as pyrimidine analogs (5-fluorouracil, floxuridine, capecitabine, gemcitabine and cytarabine) and purine analogs, folate antagonists and related inhibitors (mercaptopurine, thioguanine, pentostatin and 2-chlorodeoxyadenosine (cladribine)); antiproliferative/antimitotic agents including natural products such as vinca alkaloids (vinblastine, vincristine, and vinorelbine), microtubule disruptors such as taxane (paclitaxel, docetaxel), vincristin, vinblastin, nocodazole, epothilones and navelbine, epidipodophyllotoxins (etoposide, teniposide), DNA damaging agents (actinomycin, amsacrine, anthracyclines, bleomycin, busulfan, camptothecin, carboplatin, chlorambucil, cisplatin, cyclophosphamide, cytoxan, dactinomycin, daunombicin, doxorubicin, epirubicin, hexamethylmelamineoxaliplatin, iphosphamide, melphalan, merchlorehtamine, mitomycin, mitoxantrone, nitrosourea, plicamycin, procarbazine, taxol, taxotere, teniposide, triethylenethiophosphoramide and etoposide (VP16)); antibiotics such as dactinomycin (actinomycin D), daunorubicin, doxorubicin (adriamycin), idarubicin, anthracyclines, mitoxantrone, bleomycins, plicamycin (mithramycin) and mitomycin; enzymes (L-asparaginase which systemically metabolizes L-asparagine and deprives cells which do not have the capacity to synthesize their own asparagine); antiplatelet agents; antiproliferative/antimitotic alkylating agents such as nitrogen mustards (mechlorethamine, cyclophosphamide and analogs, melphalan, chlorambucil), ethylenimines and methylmelamines (hexamethylmelamine and thiotepa), alkyl sulfonates-busulfan, nitrosoureas (carmustine (BCNU) and analogs, streptozocin), trazenes-dacarbazinine (DTIC); antiproliferative/antimitotic antimetabolites such as folic acid analogs (methotrexate); platinum coordination complexes (cisplatin, carboplatin), procarbazine, hydroxyurea, mitotane, aminoglutethimide; hormones, hormone analogs (estrogen, tamoxifen, goserelin, bicalutamide, nilutamide) and aromatase inhibitors (letrozole, anastrozole); anticoagulants (heparin, synthetic heparin salts and other inhibitors of thrombin); fibrinolytic agents (such as tissue plasminogen activator, streptokinase and urokinase), aspirin, dipyridamole, ticlopidine, clopidogrel, abciximab; antimigratory agents; antisecretory agents (breveldin); immunosuppressives (cyclosporine, tacrolimus (FK-506), sirolimus (rapamycin), azathioprine, mycophenolate mofetil); anti-angiogenic compounds (TNP-470, genistein) and growth factor inhibitors (vascular endothelial growth factor (VEGF) inhibitors, fibroblast growth factor (FGF) inhibitors); angiotensin receptor blocker, nitric oxide donors; anti-sense oligonucleotides; antibodies (trastuzumab); cell cycle inhibitors and differentiation inducers (tretinoin); mTOR inhibitors, topoisomerase inhibitors (doxorubicin (adriamycin), amsacrine, camptothecin, daunorubicin, dactinomycin, eniposide, epirubicin, etoposide, idarubicin and mitoxantrone, topotecan, irinotecan), corticosteroids (cortisone, dexamethasone, hydrocortisone, methylpednisolone, prednisone, and prenisolone), growth factor signal transduction kinase inhibitors; mitochondrial dysfunction inducers and caspase activators; and chromatin disruptors.

Depending on the nature of the proliferative disorder and the therapy, administration of the ERM binding agents of the invention may be continued while the other therapy is being administered and/or thereafter. Administration of the ERM binding agents may be made in a single dose, or in multiple doses. In some instances, administration of the ERM binding agents is commenced at least several days prior to the conventional therapy, while in other instances, administration is begun either immediately before or at the time of the administration of the conventional therapy.

In certain therapeutic applications, the ex vivo-derived inhibitors are utilized in a manner appropriate for therapy in general. For such therapy, the inhibitors or vectors encoding inhibitors of the invention can be formulated for a variety of modes of administration, including systemic and topical or localized administration. In such embodiments, a polypeptide inhibitor may be combined with a pharmaceutically acceptable excipient, e.g., a non-pyrogenic excipient. Techniques and formulations generally may be found in Remmington's Pharmaceutical Sciences, Meade Publishing Co., Easton, Pa. For systemic administration, injection being preferred, including intramuscular, intravenous, intraperitoneal, and subcutaneous injection, the inhibitors of the invention can be formulated in liquid solutions, preferably in physiologically compatible buffers such as Hank's solution or Ringer's solution. In addition, the inhibitors may be formulated in solid form and redissolved or suspended immediately prior to use. Lyophilized forms are also included.

Systemic administration can also be by transmucosal or transdermal means, or the compounds can be administered orally. For transmucosal or transdermal administration, penetrants appropriate to the barrier to be permeated are used in the formulation. Such penetrants are generally known in the art, and include, for example, for transmucosal administration bile salts and fusidic acid derivatives. In addition, detergents may be used to facilitate permeation. Transmucosal administration may be through nasal sprays or using suppositories. For oral administration, the peptides are formulated into conventional oral administration forms such as capsules, tablets, and tonics. For topical administration, particularly cosmetic pharmaceutical compositions, the oligomers of the invention are formulated into ointments, salves, gels, or creams as generally known in the art.

Alternative means of administration of peptides have been developed. Sustained-release pharmaceutical compositions (Putney, et al. Nature Biotechnology 1998, 16, 153-157) are advantageous, requiring fewer administrations and, often, lower dosages. Techniques for oral delivery of peptides have been reviewed (Fasano, A. Trends in Biotechnology 1998, 16, 152-157), as have several site-specific means of peptide delivery (Pettit, D. K. et al. Trends in Biotechnology 1998, 16, 343-349). Additional techniques for therapeutic administration of peptides are known to those of skill in the art.

The teachings of all publications and patents cited herein are incorporated herein by reference.

### EXAMPLES

The following examples are for illustrative purpose only, and should in no way be construed to be limiting in any respect of the claimed invention.

### Example 1

### Immuno-Responsive ERM Protein is Detected in Ascitic Fluid from Ovarian Cancer Patients

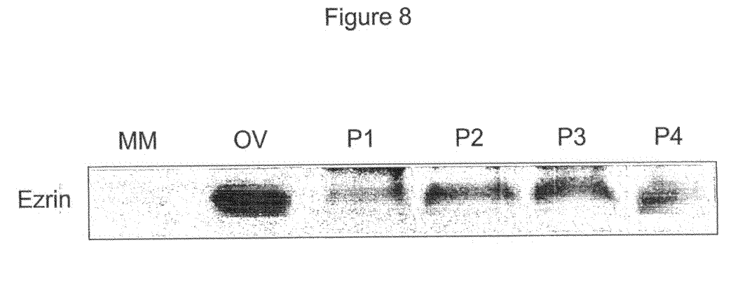

To determine if ERM proteins are shed from the surface of diseased cells and appear in body fluids, ascitic fluid from four patients with metastatic OVCA were obtained by paracentesis. The samples were immediately centrifuged, and the supernatants were snap frozen in liquid nitrogen. The samples were stored in the Yale Discovery to Cure tissue and fluid bank for an extended period of time, before they were thawed and diluted 6-8 fold (because of overloading by neat samples), and studied by Western blotting using anti-ezrin antibody (FIG. 8).

It was readily seen from the Western blot that all patients' ascitic fluids contained large amounts of immuno-reactive ezrin (ir-ezrin).

### Example 2

### Ezrin Expression is Correlated with Cancer Progression

Using Immunohistochemistry, it was found that ezrin is over-expressed in OVCA cells but not in normal ovary or in the superficial ovarian epithelial cells. In addition, this expression level was much higher in ascitic cells from patients with metastatic OVCA (data not shown). The staining of ir-ezrin was found at the base of protuberances and along the cytoplasmic edge of the ruffles, and also at the intercellular bridges. All of these stainings are characteristic of the role of ezrin in cell membrane specialization.

This experiment indicates that ezrin expression is correlated with cancer progression, and ERM protein expression is higher in cancer cells than in normal cells, and highest in metastatic cancer cells.

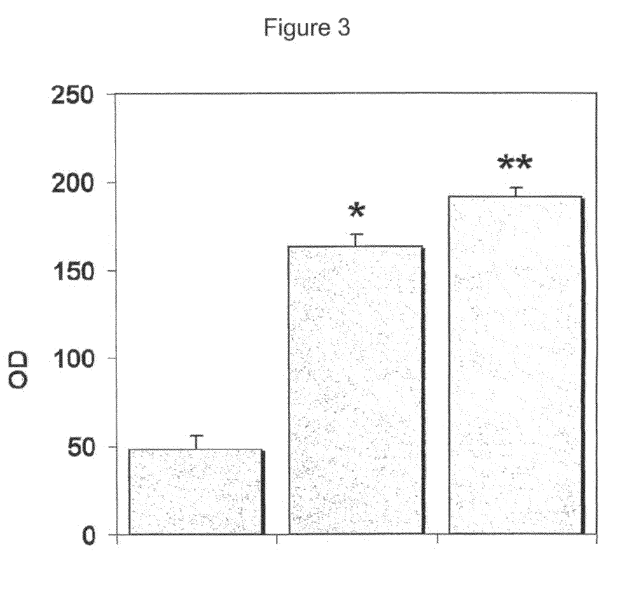

Western blot analysis also confirmed this finding. The relative expression level of ezrin was measured in protein samples from OVCA patients at different stages of cancer progression (e.g., primary cancer vs. metastatic cancer), as compared to normal ovary (FIG. 3).

For a typical Western blot analysis, approximately 200 mg of tissues kept frozen at −80° C. are homogenized and then lysed at 4° C. using 0.5 ml of ice-cold RIPA buffer (0.1% SDS, 1% Triton X-100, 1% deoxycholate, 0.15 M NaCl, 2 mM EDTA, 25 mM Tris, pH 7.5) containing 4 mg/ml of protease inhibitor cocktail tablet (Boehringer-Mannheim, Indianapolis, Ind., USA). The lysates are centrifuged at 105,000×g for 30 min at 4° C. into particulate and cytosolic fractions. The particulate pellet is resuspended in the same volume as cytosolic fractions. The equal amount of protein (26 μg) from each sample is subjected to 10% sodium dodecyl sulfate-polyacrylamide gel electrophoresis (SDS-PAGE) and transferred onto polyvinyl diflouride membranes (Millipore, Bedford, Mass., USA) in a solution containing 25 mM Tris, 92 mM glycine, 0.01% SDS, and 20% methanol. After blocking with 6% skim milk, the membranes are probed with B22 or β-tubulin (Vector Labs) for 1 h at room temperature. β-Tubulin (or other proteins such as actin) may be used as an internal control. The immunoreactive proteins are visualized using peroxidase-conjugated secondary antibodies (Vector Labs) and the ECL-chemiluminescence Western blot analysis kit (Amersham, Arlington Heights, Ill., USA). Protein quantitation is determined by the Bicinchoninic acid protein assay kit (Pierce, Rockford, Ill., USA). Analyses are performed in five independent series. The signal intensity of each band is analyzed using NIH Image software. The expression levels are determined as ratios of band intensity between ezrin and the reference protein (β-tubulin in this case) to correct for variation in the amounts of protein, and are statistically compared by Mann-Whitney U-test.

It was clear that ezrin expression was much higher (e.g., at least three-fold higher) in primary OVCA than in normal ovary, and even higher (e.g., close to four-fold higher) in metastatic OVCA. There was a clear correlation between ezrin expression and cancer progression. The differences were statistically significant.

### Example 3

### ERM Antibody Inhibits Matrigel Invasion by Metastatic Endometrial Carcinoma Cells in a Dose-Dependent Manner

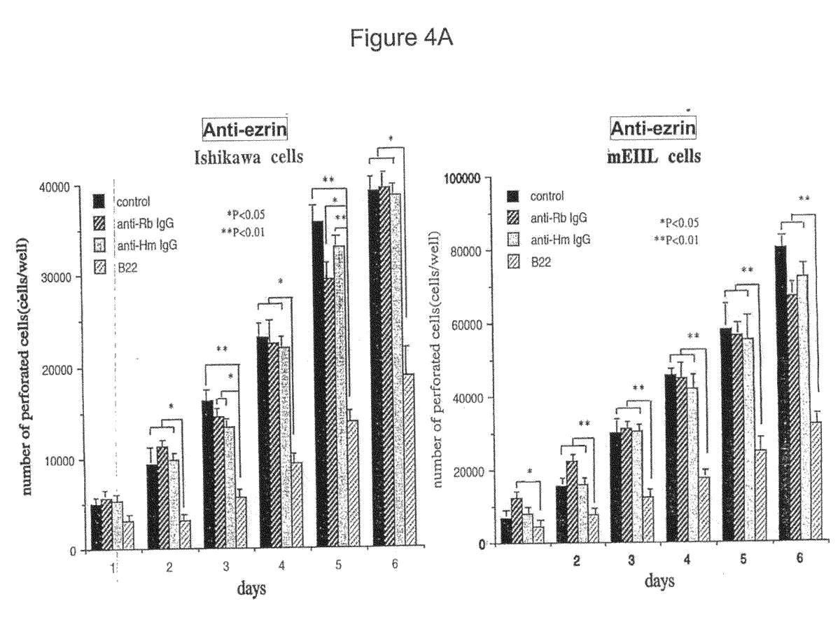

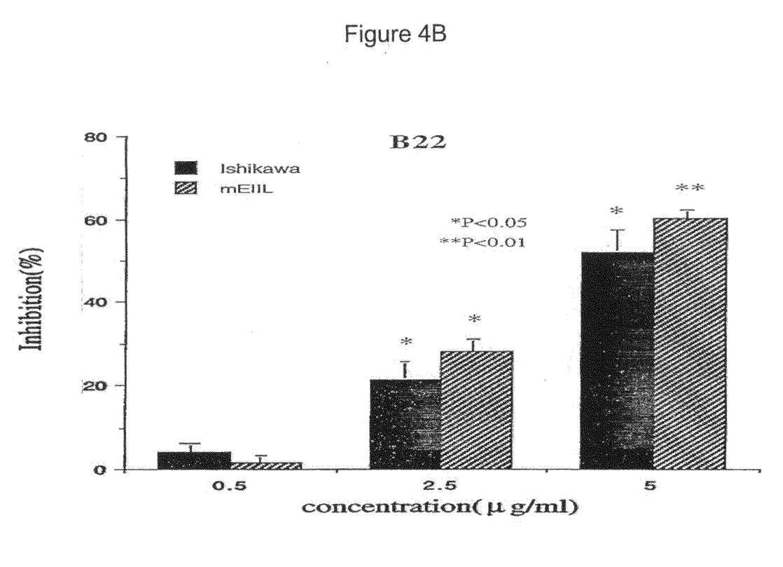

It has been previously shown that ezrin antisense polynucleotides inhibited invasion of highly-metastatic endometrial carcinoma cells in the Matrigel membrane cell invasion assay in proportion to ezrin expression, although those antisense polynucleotides did not appear to affect cancer cell proliferation. It is shown herein that ezrin antibody had surprisingly the same effect as the ezrin antisense polynucleotides in inhibiting cell invasion in the Matrigel assay (FIG. 4A).

In one experiment, an antigen-affinity-purified rabbit antiserum (polyclonal antibody “B22”) to human placental ezrin was used, which was obtained as a gift from Dr Anthony Bretscher (Cornell University, Ithaca, N.Y., USA) (Khanna et al., Cancer Res. 61: 3750-3759, 2001). B22 clearly inhibited ENDOCA cell invasion in the Matrigel assay (FIG. 4A). This antiserum does not recognize the related proteins moesin and radixin (Franck et al., J. Cell Sci. 105: 219-231, 1993).

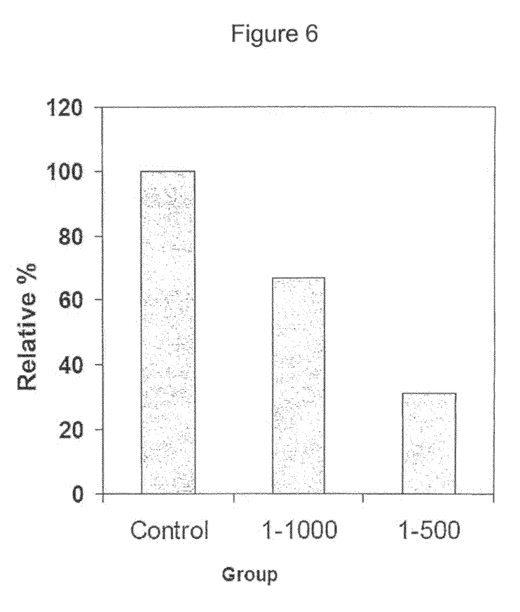

The same results for Matrigel penetration were also obtained using a commercially available antibody from Sigma (Product No. E 8897, Sigma-Aldrich, St. Louis, Mo.), in both Ishikawa cells (ENDOCA cells) and SKOV3 cells (an OVCA cell line, see Examples 4 and 7 below). The inhibitory effect of ezrin antibodies on cell growth was also seen in these experiments (see FIG. 6). In addition, the use of different titers was performed. All results were dose-responsive (see FIG. 6).

Specifically, cancer cells incubated with the B22 antibody showed diminished ability to penetrate the Matrigel, such that by Day 2 of the assay, the difference between the B22 treated sample and the control samples are statistically significant (FIG. 4A).

Incubating the highly metastatic cancer cells at the presence of as low as 2.5 μg/mL of the B22 antibody was sufficient to cause the statistically significant difference in this assay (FIG. 4B).

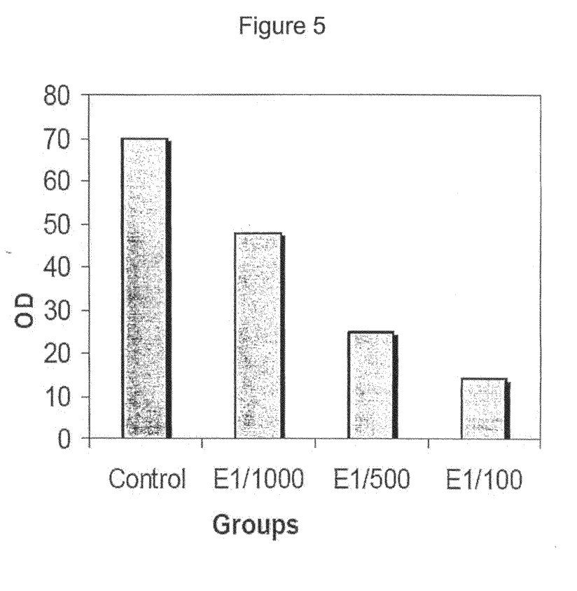

Importantly, it is clearly shown herein that the anti-invasive effect of anti-ezrin antiserum is dose-dependent (FIG. 4B). In FIG. 5, an anti-ezrin monoclonal antibody was diluted 1:100, 1:500, or 1:1000, before it was used in the Matrigel invasion assay using OVCA cells. A clear dose-dependent response was observed compared to the control assay.

These experiments unequivocally demonstrated the surprising finding that ERM binding agents (e.g., antibodies), when administered to living cells, can inhibit the function of their target molecules in a dose-dependent manner, despite the fact that such target molecules have no extracellular domain.

For a typical Matrigel invasion assay, cells are cultured for 24 hrs with serum-free OptiMEM 1×. A 0.5 ml suspension of 2.5×10⁴ cells is then layered in the upper compartment of a Boyden Chamber (BD Bioscience, Bedford, Mass., USA). The chamber has a polycarbonate filter (8 μm pore size) pre-coated with 20 μg of Matrigel basement membrane. The cells are then incubated for a further 24 h at 37° C. with serum-free OptiMEM 1×. Other reagents, such as antibodies or other inhibitors, stimulators may be incubated with the cells to test their ability to affect Matrigel invasion.

Cells that are motile and invade through the Matrigel membrane into the lower chamber are counted, and their numbers compared with a timed-vehicle control group. At the end of the incubation the Matrigel is scraped away and the remaining plastic filters are harvested and stained with the Diff-Quick stain set (DADE HEHRING AG, Dudingen, Switzerland) in order to count the cells that have penetrated the Matrigel membrane. All incubations are done in triplicate. All experiments are performed at least three times.

The Ishikawa cell line used in this study is an estrogen-dependent, well differentiated endometrial adenocarcinoma cell line. It was a gift from Dr. Nishida, Department of Obstetrics and Gynecology, Tsukuba University School of Medicine (Nishida et al., Acta Obstet. Gynaecol. Jpn. 37: 1103-1111, 1985). An estrogen independent metastatic subclone of the Ishikawa line (mEIIL: metastatic Estrogen-Independent Ishikawa Line) was established in the Department of Obstetrics and Gynecology, Nihon University School of Medicine, by culturing Ishikawa cells in estrogen-free medium over 400 days, followed by in vivo clonal selection (Sakamoto et al., Acta Obstet. Gynaecol. Jpn. 47: 249-256, 1995). Ishikawa and mEIIL cells were maintained in Eagle's MEM (basal medium; Sigma Chemical Co., St. Louis, Mo.) supplemented with 10% heat-inactivated fetal calf serum (FCS, Gibco BRL, Grand Island, N.Y.) and 1% penicillin/streptomycin/fungizone (Sigma Chemical Co.) at 37° C. in a humidified 5% CO₂ atmosphere. The culture medium was changed every 7 days. Four days prior to the experiments, cell cultures were changed from basal medium to phenol red-free mixture of Ham's F-12 and Dulbecco's modified Eagle's medium (Sigma Chemical Co.) supplemented with 5% FCS stripped of steroids by a dextran-coated charcoal treatment (experimental medium). Two days later, the cells were plated into 9 cm dishes (Corning, Corning, N.Y.) and used for all experiments.

Both mEIIL cells and Ishikawa cells were similarly tested in this assay, and both yielded similar results. The three negative controls for each experiment included no antibody control, anti-rabbit antisera control, and anti-human antisera control. Statistically significant differences are represented by “*” (P <0.05) or “**” (P<0.01).

### Example 4

### ERM Antibody Inhibits OVCA Cell Proliferation

To demonstrate the effect of ERM antibodies on OVCA proliferation, unaggregated cells were suspended by passing the cells through a 21G needle. To obtain cell adherence on the pre-coated plastic dishes, 200 μl of cell suspension (5×10³-5×10⁴ cells) was pipetted into each well of a 96 well flat-bottomed micro-plate (Becton-Dickinson scientific, NJ) and incubated for 48 h. SKOV3 cells were cultured in McCoy medium (supplemented with 10% FBS) while DOV13 and OVCA ascites cells were cultured in MEM (supplemented with 10% FBS). There was then an optional 24 h pre-incubation with serum-free OptiMEM 1×. The medium was changed to that containing various dilutions of ERM antibodies or other test agents for an additional 24-48 h.

The living cell mass was determined using the MTT assay as previously described (Song et al., J. Clin. Invest. 106: 1209-1220, 2000; Song et al., Mol. Hum. Reprod. 8: 447-455, 2002): 20 μl of MTT {3-(4,5-diethylthiazol-2-yl)-2,5-diphenyltetrazolium bromide; Sigma, St Louis) dye was added to each well 4 h before the end of the incubation. The wells were decanted, and 100 μl of acidified isopropyl alcohol was added to solubilize the reactive crystals. An automatic micro-plate reader (Model 550; Bio-Rad, Hercules, Calif.) was used to measure absorbency at 540 nm. All the tests were accompanied by a Cell Titer assay (CellTiter 96 Aqueous, Promega, Madison, Wis., USA) following the manufacture's manual.

FIG. 6 shows that anti-ezrin mAb also inhibits OVCA cell growth. (e.g., increase in cell number) by the same starting cells that are in the above figures. These cells are grown separately, in flasks, to assess cell growth or increase in cell number. The number of cells in the flasks at the end of a 24-hour period are counted. The results from cells treated with 1:1000 or 1:500 dilutions of anti-ezrin mAb are compared with that of the control untreated cultures. At the end of the experiment, the control group had four times as many cells as the 1:500 dilution of anti-ezrin mAb (e.g., about 75% inhibition). The decrease in cell number in mAb-treated culture may be due to inhibited cell proliferation or increased apoptosis, or both.

### Example 5

### ERM Protein Antibody Binds to OVCA Cells in Culture

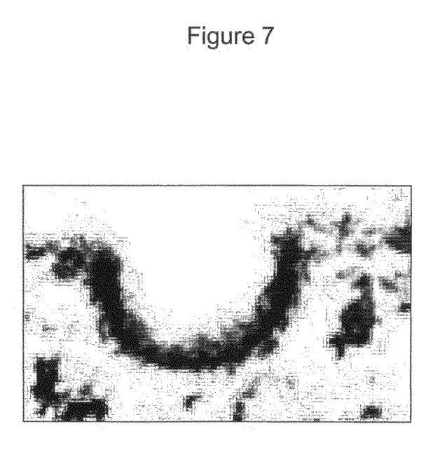

Results also demonstrated that exposure of ezrin-expressing ovarian cancer cells to a commercially available mAB against recombinant ezrin results in the binding of the mAB to live cells (FIG. 7). Control anti-actin (intracellular protein) mAB (negative control) showed only occasional binding to the cells, and control anti-FAS Receptor (cell surface antigen) mAB (positive control) bound well to the cells, indicating that the study is a proper demonstration of the binding of anti-ezrin mAB to the ezrin in the area of the cell membrane. These studies are therefore confirmatory of the finding that antiserum B22 blocked ezrin action in cancer cells.

In this experiment, OVCA cells in culture were exposed anti-exrin mAb for only 5 minutes to avoid endocytosis of the mAb. The cells were then washed, fixed, and stained using the appropriate detection antibodies and the DAB reaction.

### Example 6

### ERM Proteins are Present in Cell Membranes and in Fragments of Ezrin-Positive Cells

Electron microscopy was used to determine definitively whether the anti-ezrin mAB was bound to a membrane or an extracellular site. While studies showed the presence of free-floating, ezrin-positive material, the intact cells only have intracellular ezrin. Furthermore, following administration to the cultures, the living cells took up the anti-ezrin antibody, which was only found in the actual membranes (see above and FIG. 7). These results further indicate that ezrin is restrained to being in or near the surface of the membrane, and this is where the antibodies encounter ezrin. On the other hand, the presence of the ir-ezrin-positive fragments explains the presence of ezrin in body cavity fluids, and indicates that ezrin may be founds in body fluids in addition to ascitic fluid. These findings indicate that ERM proteins may serve as clinical tumor markers.

### Example 7

### ERM Antibodies Inhibit Tumor Xenograph Invasion/Metastasis

Immuno-compromised SCID mice are injected with about 5×10⁶ SKOV3 OVCA cells intraperitoneally to allow cancer cells to proliferate and metastasize. It has been previously shown that commercially available anti-ezrin antibodies can inhibit SKOV3 cell proliferation, as well as Matrigel invasion in the Matrigel penetration assay.

The SKOV3 cells are purchased from American Type Culture Collection (ATCC, Rockville, Md., USA). Prior to study, cells are maintained in McCoy medium or minimal essential medium (MEM) supplemented with penicillin, streptomycin, and 10% fetal calf serum (GIBCO BRL, Gaithersburg, Md., USA). Cells are cultured in 5% CO2 and humidified air and the medium was changed twice weekly. After flask-incubation to sub-confluence (60-70%) in McCoy medium or MEM containing 10% FBS, the medium is changed to phenol red-free, serum-free OptiMEM 1×.

One group of injected mice are used as control, and receive only control vehicle injection. The other similarly injected mice (the experimental group) are treated with injections of species compatible anti-ezrin mAb. Animals are assessed for rate of implantation and amount of tumor growth using standard methods starting four days after the injection of cancer cells to determine whether the treated mice have less and slower cancer growth, with fewer or no distant metastasis in the lung compared to the control mice.

A full mAb dose-response curve is also obtained to facilitate the determination of the right dose of antibody to be used in the following experiments.

The selected dose of mAb (from above) is then used to treat established OVCA. This is done by breeding transgenic mice that express the SV40 large T antigen under the transcriptional control of a portion of the 5′ upstream region (5′-UTR) of the Mullerian Inhibitory Substance type II receptor (MSIIR) gene (the TgMISIIR-TAg mice). It is expected that about 50% of the females spontaneously develop an ovarian epithelial carcinoma that is similar in histology and in its metastatic route to human OVCA. These tumors also over-express ezrin, which is confirmed by Western Blotting.

The mice are observed for the presence of tumor mass using MRI, and then treated by intra-peritoneal anti-ezrin mAb treatment. Control group mice receive vehicle treatment only. The animals normally expire within four weeks of the presence of demonstrable tumor, and thus they are sacrificed at either four weeks or six weeks. Tumor burden and mice longevity are assessed at the end point.

The female TGMISIIR-TAg mice develop bilateral ovarian tumors with spread to peritoneal organs and the presence of ascites. They typically succumb to disease with an average latency of 140 days. Thus these females are infertile. Thus to breed this line of transgenic mice, it is necessary to mate the male transgenic TgMISIIR-TAg mice with wild type females to create a breader clone.

### Example 8

### Sandwich Assay for ERM protein Detection/Quantitation

A sandwich ELISA assay is used to detect and/or quantitate ERM protein in tissue sample/fluids. For example, to detect/quantitate ezrin in a sample, binding agents such as an ezrin capture antibody is bound to a 96-well plastic plate (or other solid support). Ezrin in samples is then captured and then detected/quantitated by a specific antibody. The third element in the “sandwich” is a species-specific anti-IgG that is labeled with an enzyme, such as peroxidase. The peroxidase reaction is developed and quantitated by an ELISA plate reader.

Samples for this assay may be furnished from any source, such as by the Yale University “Discovery to Cure” cancer specimen bank. Recovery of added ascitic fluid ir-ezrin may also be preformed.

Following standardization and establishment of reliability criteria, the assay is applied to ascitic fluid obtained at diagnostic abdominal endoscopy for infertility of unknown origin (“negative control”). Further testing may be performed on fluids and blood from patients undergoing surgery or radiation/chemotherapy for OVCA/PPC and using the same materials from the Yale “Discovery to Cure” cancer specimen bank. A similar strategy will be employed for other cancers and ezrin-related diseases.

The Ovarian Cancer Tissue Bank (OCTB) is located in the Department of Obstetrics, Gynecology, and Reproductive Sciences of Yale University, and contains approximately 500 tissue samples of primary and metastatic ovarian cancers as well as tissue samples from normal ovaries. In addition, as part of the NCI Ovarian Cancer Detection Program, the facility has in storage ascites and serum samples from patients with ovarian cancer and normal age matched controls. An important component of the Tissue Bank is its panel of ovarian cancer cells (n=36) isolated from ascites and 10 immortalized normal Ovarian Surface Epithelial cells (OSE). The panel is regularly used for the screening of new compounds that may have cytotoxic effects on ovarian cancer.

### Equivalents

Those skilled in the art will recognize, or be able to ascertain using no more than routine experimentation, numerous equivalents to the specific method and reagents described herein, including alternatives, variants, additions, deletions, modifications and substitutions. Such equivalents are considered to be within the scope of this invention and are covered by the following claims.

1. A method of inhibiting a proliferative condition in an individual, comprising administering to the individual an effective amount of a binding agent which binds an ERM family protein.

2. The method of claim 1, wherein the proliferative condition is cancer.

3. The method of claim 2, wherein the cancer is ovarian cancer, endometrial cancer (endometrial adenocarcinoma, such as UEC), primary peritoneal cancer (PPC), renal adenocarcinoma, brain hemangioblastoma, pancreatic adenocarcinoma, epidermoid carcinoma, osteosarcoma, epithelial cancer, melanoma, squamous skin carcinoma, leukemia, breast cancer, glioblastoma, schwannoma, meningioma, malignant mesothelioma, neurofibromatosis, colon cancer, oral cancer, or rhabdomyosarcoma.

4-6. (canceled)

7. The method of claim 1, wherein the proliferative condition is a benign proliferative disorder.

8. The method of claim 7, wherein the proliferative condition is tuberosclerosis, psoriasis, endometriosis, complex endometrial hyperplasia (cH), atypical endometrial hyperplasia (aH), polyps, or neurofibromatosis.

9. The method of claim 1, wherein the individual is a human or a non-human mammal.

10. The method of claim 1, wherein the binding agent is an antibody, or a functional fragment thereof.

11-16. (canceled)

17. The method of claim 1, wherein the binding agent inhibits the interaction of the ERM family protein with a cell surface receptor.

18. (canceled)

19. The method of claim 1, wherein the ERM family protein is ezrin.

20. The method of claim 1, further comprising administering a second therapeutic agent.

21. (canceled)

22. A method of diagnosis of a proliferative condition in an individual, comprising determining the amount and/or concentration of an ERM family protein in a body fluid sample from an individual suspected of having or at risk of having the proliferative condition, wherein an amount and/or concentration of the ERM family protein significantly higher in the body fluid sample from the individual than in a normal or control sample is indicative of the existence of the proliferative condition in the individual.

23. A method of monitoring in an individual the progress or recurrence of a proliferative condition, comprising determining the amount and/or concentration of an ERM family protein in a body fluid sample from an individual who has or had the proliferative condition, wherein an amount and/or concentration of the ERM family protein significantly higher in the body fluid sample from the individual than that in a normal or control sample is indicative of the progress or recurrence of the proliferative condition in the individual.

24-25. (canceled)

26. The method of claim 22, wherein the amount and/or concentration of the ERM family protein in the sample is proportionally indicative of the severity and/or extent of the proliferative condition.

27. The method of claim 22, wherein the amount and/or concentration of the ERM family protein is used along with the results of one or more diagnostic tests selected from the group consisting of: mammography, an early mammography program, a frequent mammography program, a biopsy procedure using a tissue of the individual, an ultrasound analysis of a suspected disease organ and optionally a normal organ, a magnetic resonance imaging (MRI) analysis of a suspected disease organ and optionally a normal organ, an electrical impedance (T-scan) analysis of a suspected disease organ and optionally a normal organ, ductal lavage, a nuclear medicine analysis, sequence analysis of one or more disease-associated genes, and a thermal imaging of a suspected disease organ and optionally a normal organ.

28. The method of claim 22, wherein the proliferative condition is cancer.

29. The method of claim 28, wherein the cancer is ovarian cancer, endometrial cancer (ENDOCA), endometrial adenocarcinoma (e.g., UEC), primary peritoneal cancer (PPC), renal adenocarcinoma, brain hemangioblastoma, pancreatic adenocarcinoma, epidermoid carcinoma, osteosarcoma, epithelial cancer, melanoma, squamous skin carcinoma, leukemia, breast cancer, glioblastoma, schwannoma, meningioma, malignant mesothelioma, neurofibromatosis, colon cancer, oral cancer, or rhabdomyosarcoma.

30. (canceled)

31. The method of claim 22, wherein the proliferative condition is a benign proliferative disorder.

32. The method of claim 31, wherein the proliferative condition is wherein the proliferative condition is tuberosclerosis, psoriasis, endometriosis, complex endometrial hyperplasia (cH), atypical endometrial hyperplasia (aH), polyps, or neurofibromatosis.

33. The method of claim 22, wherein the individual is a human or a non-human mammal.

34. The method of claim 22, wherein the amount and/or concentration of the ERM family protein is determined using a binding agent which binds the ERM family protein.

35. The method of claim 34, wherein the binding agent is an antibody, or a functional fragment thereof.

36-58. (canceled)
